(19) United States
(12) Patent Application Publication
(10) Pub. No.: US 2007/0142392 A1
(43) Pub. Date: Jun. 21, 2007

(54) Uses of rifamycins

(75) Inventors: Christopher K. Murphy, Upton, MA (US); David M. Rothstein, Lexington, MA (US)

Correspondence Address: CLARK & ELBING LLP, 101 FEDERAL STREET, BOSTON, MA, 02110, US

(73) Assignee: ActivBiotics, Inc., Lexington, MA (US)

(21) Appl. No.: 11/638,738

(22) Filed: Dec. 14, 2006

Related U.S. Application Data
(60) Provisional application No. 60/750,774, filed on Dec. 15, 2005.

(51) Int. Cl. A61K 31/496; A61K 38/14; A61K 31/7048; A61K 31/7034; A61K 31/65; A61K 31/43; A61K 31/545; A61K 31/57

(52) U.S. Cl. 514/252.13; 514/8; 514/28; 514/29; 514/35; 514/37; 514/39; 514/192; 514/200; 514/254.08; 514/152; 514/156; 514/171; 514/210.09

(57) ABSTRACT

The invention features methods, compositions, and kits for treating prosthetic joint infections, foreign body infections, infectious arthritis, and osteomyelitis

### CROSS-REFERENCE TO RELATED APPLICATIONS

This application claims the benefit of U.S. Provisional Application No. 60/750,774, filed Dec. 15, 2005, hereby incorporated by reference.

### BACKGROUND OF THE INVENTION

The present invention relates to the field of antimicrobial agents.

Arthroscopy (joint replacement surgery) is the major procedure to alleviate pain and to improve mobility in people with damaged joints. Infections associated with prosthetic joints are significant complications with high morbidity and substantial costs. In addition to protracted hospitalization, patients risk complications associated with additional surgery and antimicrobial treatment, as well the possibility of renewed disability.

The incidence of infection depends on the type of prosthesis. According to one report, in a study involving hip and knee prostheses, the incidence of infection was 5.9 per 1000 prosthesis-years during the first 2 years after implantation and 2.3 per 1000 prosthesis-years during the following 8 years. The incidence of prosthetic joint infections will likely increase due to (i) better detection methods for microbial biofilms involved in prosthetic joint infections, (ii) the growing number of implanted prostheses in the ageing population, and (iii) the increasing residency time of prostheses, which are at continuous risk for infection during their implanted lifetime.

Other medical implants are also accompanied with a risk of infection. The presence of a medical implant increases the pathogenic potential of bacteria. Many medical devices transect cutaneous barriers and thus provide a direct route of bacterial invasion. Many implants are coated by a film of proteins such as fibronectin, fibrin, and laminin. Fibronectin plays a crucial role in promoting initial staphylococcal attachment. In addition, subcutaneous implants have been shown to impair the phagocytic-bacteriocidal capacity of local granulocytes.

There is a need for improved methods for treating infections associated with prosthetic joints and other medical implants.

### SUMMARY OF THE INVENTION

The invention features methods, compositions, and kits for treating prosthetic joint infections, foreign body infections, infectious arthritis, and osteomyelitis. Rifamycins that are useful in the methods, compositions, and kits of the invention are described by formulas (I)-(V).

In one aspect, the invention features a method for treating a prosthetic joint infection in a patient in need thereof by administering to the patient a rifamycin of any one of formulas (I)-(V) (e.g., a compound described in Tables 1-4) in an amount effective to treat the prosthetic joint infection.

The invention also features a method for treating a foreign body infection in a patient in need thereof by administering to the patient a rifamycin of any one of formulas (I)-(V) in an amount effective to treat the foreign body infection in the patient.

The invention also features a method for treating infectious arthritis in a patient in need thereof by administering to the patient a rifamycin of any one of formulas (I)-(V) in an amount effective to treat the infectious arthritis in the patient.

The invention also features a method for treating osteomyelitis in a patient in need thereof by administering to the patient a rifamycin of any one of formulas (I)-(V) in an amount effective to treat the osteomyelitis in the patient.

In any of the foregoing aspects, the dosage of the rifamycin is normally about 0.001 to 1000 mg/day. The compound may be given daily (e.g., a single oral dose of 2.5 to 25 mg/day) or less frequently (e.g., a single oral dose of 5, 12.5, or 25 mg/week). Treatment may be for one day to six months, nine months, one year, or longer. In one embodiment, the rifamycin is administered at an initial dose of 2.5 to 100 mg for one to seven consecutive days, followed by a maintenance dose of 0.005 to 10 mg once every one to seven days for one month, one year, or even for the life of the patient.

If desired, a rifamycin may be administered in conjunction with one or more additional antibacterial agents (e.g., sulfonamides, tetracyclines, aminoglycosides, macrolides, lincosamides, ketolides, fluoroquinolones, glycopeptide antibiotics, and polymyxin antibiotics) such as azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, or trimethoprim). Particularly suitable antibiotics for treating prosthetic joint infections are quinolones (e.g., moxifloxacin, levofloxacin, gatifloxacin, ciprofloxacin, fleroxacin, and ofloxacin), cotrimoxazole (trimethoprim and sulfamethoxazole), minocycline, fusidic acid, linezolid, nafcillin, teicoplanin, penicillin G, ceftriaxone, ceftazidime, cefepime, clindamycin, amoxicillin, ampicillin, carbapenem, and daptomycin. These additional agents may be administered within 14 days, 7 days, 1 day, 12 hours, or 1 hour of administration of a rifamycin, or simultaneously therewith. The additional therapeutic agents may be present in the same or different pharmaceutical compositions as the rifamycin. When present in different pharmaceutical compositions, different routes of administration may optionally be used. For example, a rifamycin may be administered orally, while a second agent may be administered by intravenous, intramuscular, or subcutaneous injection.

The invention also features an orthopedic implant which releases a rifamycin of any one of formulas (I)-(V) and, optionally, a second antibiotic, such as one described herein. The implant can be covered or coated in whole or in part with a composition comprising the rifamycin. This composition may further include a biodegradable or non-biodegradable polymer.

The invention also features other types of medical implants which release a rifamycin of any one of formulas (I)-(V), such as vascular catheters, prosthetic heart valves, cardiac pacemakers, implantable cardioverter defibrillators, vascular grafts, ear, nose, or throat implants, urological implants, endotracheal or tracheostomy tubes, dialysis catheters, CNS shunts, and ocular implants.

The invention also features a composition that includes a polymer and a rifamycin of any one of formulas (I)-(V). The polymer may be a biodegradable or a non-biodegradable polymer.

The invention also features a method for reducing or inhibiting infection associated with a medical implant by introducing into a patient a medical implant that has been covered or coated with a rifamycin of any one of formulas (I)-(V) and, optionally, a second antibiotic.

The invention also features a method for making a medical implant by covering or coating a medical implant with a rifamycin of any one of formula (I)-(V). In one embodiment, the medical implant is covered or coated with the rifamycin by dipping or by impregnation.

The invention also features kits for use in treating prosthetic joint infections, infectious arthritis, osteomyelitis, and foreign body infections. One such kit includes (a) a rifamycin of any one of formulas (I)-(V); and (b) instructions for administering the rifamycin and, optionally, a second antibiotic, to a patient having a prosthetic joint infection, infectious arthritis, osteomyelitis, or a foreign body infection. Another kit includes: (a) a rifamycin of any one of formulas (I)-(V); (b) a second antibiotic; and (c) instructions for administering the rifamycin and the second antibiotic to a patient having a prosthetic joint infection, infectious arthritis, osteomyelitis, or a foreign body infection. A third kit includes: (a) a composition containing a rifamycin of any one of formulas (I)-(V) and a second antibiotic; and (b) instructions for administering the composition to a patient having a prosthetic joint infection, infectious arthritis, osteomyelitis, or a foreign body infection.

By “effective amount” is meant the amount of a compound required to treat or prevent an infection. The effective amount of active compound(s) used to practice the present invention for therapeutic or prophylactic treatment of conditions caused by or contributed to by a microbial infection varies depending upon the manner of administration, the age, body weight, and general health of the subject. Ultimately, the attending physician or veterinarian will decide the appropriate amount and dosage regimen. Such amount is referred to as an “effective” amount.

The term “administration” or “administering” refers to a method of giving a composition of the invention to a patient, by a route such as inhalation, ocular administration, nasal instillation, parenteral administration, dermal administration, transdermal administration, buccal administration, rectal administration, sublingual administration, perilingual administration, nasal administration, topical administration, and oral administration. Parenteral administration includes intrathecal, intraarticular, intravenous, intraperitoneal, subcutaneous, and intramuscular administration. The optimal method of administration of a drug or drug combination to treat a particular disease can vary depending on various factors, e.g., the oral bioavailability of the drug(s), the anatomical location of the disease tissue, and the severity of disease.

By “treat” is meant to administer a pharmaceutical composition for prophylactic and/or therapeutic purposes, wherein the growth of bacteria is prevented, stabilized, or inhibited, or wherein bacteria are killed.

The terms “animal,” “subject,” and “patient” specifically include humans, cattle, horses, dogs, cats, and birds, but also can include many other species.

As used herein, the terms “alkyl” and the prefix “alk-” are inclusive of both straight chain and branched chain saturated or unsaturated groups, and of cyclic groups, i.e., cycloalkyl and cycloalkenyl groups. Unless otherwise specified, acyclic alkyl groups are from 1 to 6 carbons. Cyclic groups can be monocyclic or polycyclic and preferably have from 3 to 8 ring carbon atoms. Exemplary cyclic groups include cyclopropyl, cyclopentyl, cyclohexyl, and adamantyl groups. Alkyl groups may be substituted with one or more substituents or unsubstituted. Exemplary substituents include alkoxy, aryloxy, sulfhydryl, alkylthio, arylthio, halogen, alkylsilyl, hydroxyl, fluoroalkyl, perfluoralkyl, amino, aminoalkyl, disubstituted amino, quaternary amino, hydroxyalkyl, carboxyalkyl, and carboxyl groups. When the prefix “alk” is used, the number of carbons contained in the alkyl chain is given by the range that directly precedes this term, with the number of carbons contained in the remainder of the group that includes this prefix defined elsewhere herein. For example, the term “C₁-C₄ alkaryl” exemplifies an aryl group of from 6 to 18 carbons attached to an alkyl group of from 1 to 4 carbons.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

By “heteroaryl” is meant an aromatic ring or ring system that contains at least one ring hetero-atom (e.g., O, S, Se, N, or P). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, tetrazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

By “heterocycle” is meant a non-aromatic ring or ring system that contains at least one ring heteroatom (e.g., O, S, Se, N, or P). Unless otherwise specified, heterocyclic groups are from 2 to 9 carbons. Heterocyclic groups include, for example, dihydropyrrolyl, tetrahydropyrrolyl, piperazinyl, pyranyl, dihydropyranyl, tetrahydropyranyl, dihydrofuranyl, tetrahydrofuranyl, dihydrothiophene, tetrahydrothiophene, and morpholinyl groups.

Aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, alkaryl (where the alkyl group has from 1 to 4 carbon atoms) and alkheteroaryl (where the alkyl group has from 1 to 4 carbon atoms).

By “alkoxy” is meant a chemical substituent of the formula —OR, where R is an alkyl group. By “aryloxy” is meant a chemical substituent of the formula —OR′, where R′ is an aryl group.

By “C_{x-y} alkaryl” is meant a chemical substituent of formula —RR′, where R is an alkyl group of x to y carbons and R′ is an aryl group as defined elsewhere herein.

By “C_{x-y} alkheteraryl” is meant a chemical substituent of formula RR″, where R is an alkyl group of x to y carbons and R″ is a heteroaryl group as defined elsewhere herein.

By “halide” or “halogen” or “halo” is meant bromine, chlorine, iodine, or fluorine.

By “non-vicinal O, S, or NR” is meant an oxygen, sulfur, or nitrogen heteroatom substituent in a linkage, where the heteroatom substituent does not form a bond to a saturated carbon that is bonded to another heteroatom.

In structural representations where the chirality of a carbon has been left unspecified, it is to be presumed by one skilled in the art that either chiral form of that stereocenter is possible.

By “benzoxazinorifamycin” is meant a compound described by formula (A): 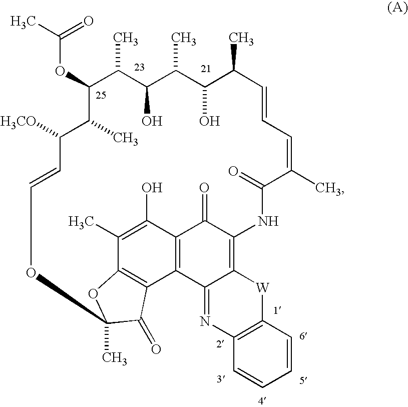
where W is O. By “benzthiazinorifamycin” is meant a compound described by formula (A), where W is S. By “benzdiazinorifamycin” is meant a compound described by formula (A), where W is N—R. For benzdiazinorifamycin, R can be H or an alkyl substituent. When R is an alkyl substituent, it is referred to as N′—R (e.g., N′-methyl) in the naming of the compound. Benzoxazinorifamycin, benzthiazinorifamycin, and benzdiazinorifamycin analogs that contain substituents are numbered according to the numbering provided in formula (A). By “25-O-deacetyl” rifamycin is meant a rifamycin analog in which the acetyl group at the 25-position has been removed. Analogs in which this position is further derivatized are referred to as a “25-O-deacetyl-25-(substituent)rifamycin”, in which the nomenclature for the derivatizing group replaces “substituent” in the complete compound name. For example, a benzoxazinorifamycin analog in which the 25-acetyloxy group has been transformed to a carbonate group, with the other side of the carbonate bonded to a 2,3-dihydroxypropyl group, is referred to as a “25-O-deacetyl-25-(2″,3″-dihydroxypropylcarbonoxy)-benzoxazinorifamycin.”

### BRIEF DESCRIPTION OF THE FIGURES

FIG. 1 is an illustration demonstrating implantation of Teflon tissue cages (32×10 mm; Novartis AG, Basel) into the flanks of guinea pigs. Cages were perforated by 130 regularly spaced holes of 1 mm diameter. Four tissue cages were implanted into albino guinea pigs weighing 700-900 g. For pharmacokinetic studies, non-infected animals were used. For antimicrobial treatment studies, cages were infected by percutaneous inoculation (200 μl) of a stationary overnight culture containing 2×10⁴ CFU S. aureus. Antimicrobial treatment was initiated 24 hours after cage infection (day 1).

FIG. 2A is a schematic illustration showing the peak drug concentration of Compound 86 (1.13 μg/ml) in cage fluid from non-infected animals after single dose of 12.5 mg/kg. Samples of cage fluid were aspirated by percutaneous cage puncture from non-infected animals at various times for 12 hours following intraperitoneal administration of 12.5 mg/Kg of Compound 86. The minimal inhibitory concentration (MIC) was determined by broth dilution method with a standard inoculum of S. aureus ATCC29213 at 5×10⁵ CFU/ml. The minimal bactericidal concentration (MBC) for logarithmic phase growth (MBC_{log}) was defined as antimicrobial concentration that reduced the original inoculum by <99.9% after 24 hour incubation (i.e. 3 log 10 CFU/ml), as described in the Manual of Clinical Microbiology (Murray et al., Manual of Clinical Microbiology). The MBC in the stationary growth phase (MBCₛₜₐₜ) was determined by using overnight bacterial cultures which were centrifuged and resuspended in medium containing 1% glucose supplemented phosphate buffered saline (PBS) pH 7.4 with 4% Muller Hinton Broth (Zimmerli et al., J Antimicrob. Chemother. 33:959-967 (1994)). In this medium, bacterial counts remained stable in the absence of antibacterial agents for >36 hours. The MIC (0.002 μg/ml), MBC_{log}, (0.008 μg/ml) and the MBCₛₜₐₜ (1.13 μg/ml) for Compound 86 are represented by the respectively labeled dotted lines.

FIG. 2B is a schematic illustration showing the peak drug concentration of rifampin (0.98 μg/ml) in cage fluid from non-infected animals after single dose of the antimicrobial. Samples of cage fluid were aspirated by percutaneous cage puncture from non-infected animals at various times for 12 hours following intraperitoneal administration of 12.5 mg/kg of rifampin. The minimal inhibitory concentration (MIC) was determined by broth dilution method with a standard inoculum of S. aureus ATCC29213 at 5×10⁵ CFU/ml. The minimal bactericidal concentration (MBC) for logarithmic phase growth (MBC_{log}) was defined as antimicrobial concentration that reduced the original inoculum by <99.9% after 24 hour incubation (i.e. 3 log 10 CFU/ml), as described in the Manual of Clinical Microbiology (Murray et al., Manual of Clinical Microbiology). The MBC in the stationary growth phase (MBCₛₜₐₜ) was determined by using overnight bacterial cultures which were centrifuged and resuspended in medium containing 1% glucose supplemented phosphate buffered saline (PBS) pH 7.4 with 4% Muller Hinton Broth. In this medium, bacterial counts remained stable in the absence of antibacterial agents for >36 hours. The MIC (0.016 μg/ml) and MBC_{log}, (0.8 μg/ml) of rifampin are represented by the respectively labeled dotted lines. The MBCₛₜₐₜ of rifampin (3.6 μg/ml), which was not reached at the peak drug concentration of rifampin, is indicated in the legend.

FIG. 3A is a schematic illustration showing the efficacy of antimicrobial treatments following infection of with S. aureus. Antimicrobial treatment was initiated 24 hours after cage infection (day 1). The eight treatment groups include: control (saline), levofloxacin 5 mg/kg, rifampin 12.5 mg/kg (with and without levofloxacin 5 mg/kg), Compound 86 at 3 mg/kg and 12.5 mg/kg (each dose with and without levofloxacin 5 mg/kg). Antibiotics were administered intraperitoneally every 12 hours for four days (total eight doses). Each antimicrobial regimen was evaluated in 12 cages (i.e., three animals with four cages each) by determining the mean reduction in the Log₁₀ CFU (+/−SD) count during the treatment before the last antimicrobial dose (day 4) or five days after completion of treatment (day 9) compared to the bacterial counts 24 h after infection immediate before initiation of treatment (day 1, ≈10⁷ CFU/ml).

FIG. 3B is a schematic illustration showing the cure rate of the antimicrobial treatments outlined in FIG. 3A. The cure rate is the fraction of cages in which the infection was eradicated. This is defined as the absence of growth of S. aureus in a TSB (trypticase soy broth) mixture containing explanted cages (removed on day 9) incubated for 24 hours at 37° C. Following incubation, 50 μl of the TSB mixture was plated on blood agar plates to determine the presence of bacteria.

### DETAILED DESCRIPTION OF THE INVENTION

The invention provides methods, compositions, and kits for treating a variety of bacterial infections, including prosthetic joint infections, infections caused by medical implants, infectious arthritis, and osteomyelitis. The methods, compositions, and kits employ rifamycins of any one of formulas (I)-(V). The methods of the invention include (i) methods of treating one of the foregoing infections by administering a rifamycin of any one of formulas (I)-(V); (ii) methods for reducing or inhibiting infection associated with a medical implant by introducing into a patient a medical implant that has been covered or coated with a rifamycin of any one of formulas (I)-(V); and (iii) methods for making a medical implant by covering or coating a medical implant with a rifamycin of any one of formulas (I)-(V). The compositions of the invention include (i) medical implants that release a rifamycin of any one of formulas (I)-(V); and (ii) compositions having a polymer and a rifamycin of any one of formulas (I)-(V). The kits of the invention include (i) kits including a rifamycin of any one of formulas (I)-(V) and instructions for administering the rifamycin, either alone or in combination with a second antibiotic, to a patient having one of the foregoing infections (or being at risk for developing one of these infections); and (ii) kits including a medical device that releases a rifamycin of any one of formulas (I)-(V) and instructions for implanting the medical device.

Treatment of Prosthetic Joint Infections

The invention provides methods, compositions, and kits for treating prosthetic joint infections following arthroplasty, including hip arthroplasty, knee arthroplasty, spinal disc arthroplasty (e.g., cervical arthroplasty, lumbar arthroplasty) proximal interphalangeal joint arthroplasty, metacarpophalangeal joint arthroplasty, arthroplasty of the thumb axis, arthroplasty of the distal radio-ulnar joint, wrist arthroplasty, shoulder arthroplasty, and elbow arthroplasty.

Infections associated with prosthetic joints cause significant morbidity. Numerous organisms are associated with prosthetic joint infections, including methicillin-sensitive and methicillin-resistant Staphylococcus aureus or coagulase-negative staphylococci such as Staphylococcus epidermis; Streptococcus spp.; Enterococcus spp.; anaerobic bacteria such as Propionibacterium acnes, Peptostreptococcus magnus, Fusobacterium spp., Clostridium spp., and Bacteroides spp.; and quinolone-sensitive Gram-negative bacilli such as Pseudomonas aeruginosa.

In one aspect, the prosthetic joint infection is treated by administering to the patient a rifamycin of any one of formulas (I)-(V) (e.g., a compound listed in one of Tables 1-4), alone or in combination with one or more additional therapies (e.g., a second antibiotic or surgical therapy.

When administered to treat a prosthetic joint infection, the dosage of the rifamycin is normally about 0.001 to 1000 mg/day. The compound may be given daily (e.g., a single oral dose of 2.5 to 25 mg/day) or less frequently (e.g., a single oral dose of 5, 12.5, or 25 mg/week). Treatment may be for one day to six months, nine months, one year, or longer. In one embodiment, the rifamycin is administered at an initial dose of 2.5 to 100 mg for one to seven consecutive days, followed by a maintenance dose of 0.005 to 10 mg once every one to seven days for one month, one year, or even for the life of the patient.

Antimicrobial Therapy

If desired, a rifamycin may be administered in conjunction with one or more additional antibiotics (e.g., azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim). Particularly suitable antibiotics for treating prosthetic joint infections are quinolones (e.g., moxifloxacin, levofloxacin, gatifloxacin, ciprofloxacin, fleroxacin, and ofloxacin), cotrimoxazole (trimethoprim and sulfamethoxazole), minocycline, fusidic acid, linezolid, nafcillin, teicoplanin, penicillin G, ceftriaxone, ceftazidime, cefepime, clindamycin, amoxicillin, ampicillin, carbapenem, and daptomycin. These additional agents may be administered within 14 days, 7 days, 1 day, 12 hours, or 1 hour of administration of a rifamycin, or simultaneously therewith.

The additional antibiotic(s) may be present in the same or different pharmaceutical compositions as the rifamycin. For example, a rifamycin may be administered intravenously or orally while a second antibiotic is administered intramuscularly, intravenously, subcutaneously, orally or intraperitoneally. The rifamycin and the second antibiotic may be given sequentially in the same intravenous line, after an intermediate flush, or may be given in different intravenous lines. The rifamycin and the second antibiotic may be administered simultaneously or sequentially, as long as they are given in a manner sufficient to allow both agents to achieve effective concentrations at the site of infection. Concurrent administration of the two agents may provide greater therapeutic effects in vivo than either agent provides when administered singly. It may permit a reduction in the dosage of one or both agents with achievement of a similar therapeutic effect. Alternatively, the concurrent administration may produce a more rapid or complete bactericidal/bacteriostatic effect than could be achieved with either agent alone.

Therapeutic effectiveness is based on a successful clinical outcome, and does not require that the antimicrobial agent or agents kill 100% of the organisms involved in the infection. Success depends on achieving a level of antibacterial activity at the site of infection that is sufficient to inhibit the bacteria in a manner that tips the balance in favor of the host. When host defenses are maximally effective, the antibacterial effect required may be minimal. Reducing organism load by even one log (a factor of 10) may permit the host's own defenses to control the infection. In addition, augmenting an early bactericidal/bacteriostatic effect can be more important than long-term bactericidal/bacteriostatic effect. These early events are a significant and critical part of therapeutic success, because they allow time for host defense mechanisms to activate. Increasing the bactericidal rate may be particularly important for joint infections.

Surgical Therapy

If desired, the rifamycin therapy can be administered in conjunction with surgical therapy, such as debridement with retention, one-stage (direct) exchange (the removal and implantation of a new prosthesis during the same surgical procedure), two-stage exchange (i.e., the removal of the prosthesis with implantation of a new prosthesis during a later surgical procedure), or permanent removal of the device.

Treatment of Infections Associated With other Implants

The invention provides methods, compositions, and kits for treating infections caused by or associated with medical implants other than prosthetic joint infections (referred to herein as “foreign body infections”). Many prosthetic or foreign devices transect cutaneous barriers, providing a direct route of bacterial invasion. Infections caused by other medical implants (e.g., intravascular devices; cardiovascular devices; neurological/neurosurgical devices; gastrointestinal devices; genitourinary devices; central venous catheters; urinary catheters; prosthetic heart valves, vascular grafts; ophthalmologic implants; otolaryngology devices; plastic surgery implants; and catheter cuffs) can be treated by administering a rifamycin of any one of formula (I)-(V), either alone or in combination with a second antibiotic, using the dosing regimens provided herein.

Implant Coatings and Biopolymers

In one embodiment, a rifamycin is formulated into a coating applied to the surface of the components of the orthopedic implant. Drugs can be applied in several manners: (a) as a coating applied to the external intraosseous surface of the prosthesis; (b) as a coating applied to the external (articular) surface of the prosthesis; (c) as a coating applied to all or parts of both surfaces; (d) as a coating applied to the surface of the orthopedic hardware (plates, screws, etc); (e) incorporated into the polymers which comprise the prosthetic joints (e.g., articular surfaces and other surface coatings) and hardware (e.g., polylactic acid screws and plates); and/or (f) incorporated into the components of the cements used to secure the orthopedic implants in place.

Drug-coating of, or drug incorporation into, an medical implant will allow bacteriocidal drug levels to be achieved locally on the implant surface, thus reducing the incidence of bacterial colonization and subsequent development of infectious complications, while producing negligible systemic exposure to the drugs. Although polymeric carriers are not required for attachment of the drug, several polymeric carriers are particularly suitable for use in this embodiment. Of particular interest are polymeric carriers such as polyurethanes (e.g., ChronoFlex AL 85A (CT Biomaterials), HydroMed640™ (CT Biomaterials), HYDROSLIP C™ (CT Biomaterials), HYDROTHANE™ (CT Biomaterials)), acrylic or methacrylic copolymers (e.g., poly(ethylene-co-acrylic acid), cellulose-derived polymers (e.g., nitrocellulose, cellulose acetate butyrate, cellulose acetate propionate), and acrylate and methacrylate copolymers (e.g., poly(ethylene-co-vinyl acetate)), polyalkylene oxides (e.g., polyethylene glycol), as well as blends thereof. The drugs of interest can also be incorporated into calcium phosphate or hydroxyapatite coatings on the medical devices.

As medical implants are made in a variety of configurations and sizes, the exact dose administered will vary with implant size, surface area, design and portions of the implant coated. However, certain principles can be applied in the application of this art. Drug dose can be calculated as a function of dose per unit area (of the portion of the implant being coated), total drug dose administered can be measured and appropriate surface concentrations of active drug can be determined.

A wide variety of implants or devices can be coated with or otherwise constructed to contain and/or release the therapeutic agents provided herein. Representative examples include cardiovascular devices (e.g., implantable venous catheters, venous ports, tunneled venous catheters, chronic infusion lines or ports, including hepatic artery infusion catheters, pacemakers and pacemaker leads, implantable cardioverter defibrillators); neurological/neurosurgical devices (e.g., ventricular peritoneal shunts, ventricular atrial shunts, nerve stimulator devices, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, and suspensions or solid implants to prevent surgical adhesions); genitourinary devices (e.g., uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy), central venous catheters, urinary catheters, peritoneal access devices); prosthetic heart valves; intravascular devices (e.g., stents, balloon catheters, autologous venous/arterial grafts, prosthetic venous/arterial grafts, vascular catheters, vascular shunts); ophthalmologic implants (e.g., moltino implants and other implants for neovascular glaucoma, drug eluting contact lenses for pterygiums, splints for failed dacrocystalrhinostomy, drug eluting contact lenses for corneal neovascularity, implants for-diabetic retinopathy, drug eluting contact lenses for high risk corneal transplants); otolaryngology devices (e.g., ossicular implants, Eustachian tube splints or stents for glue ear or chronic otitis as an alternative to transtempanic drains); plastic surgery implants (e.g., breast implants or chin implants); and catheter cuffs.

In addition to being useful for the treatment of prosthetic joint infections and foreign body infections, the rifamycins described herein can be used to treat bone and joint infections generally, including acute and chronic infectious arthritis, and acute and chronic osteomyelitis.

Treatment of Infectious Arthritis

The invention provides methods, compositions, and kits for treating infectious arthritis (e.g., acute infectious arthritis or chronic infectious arthritis). The infectious arthritis can be treated by administering to the patient a rifamycin of any one of formulas (I)-(V) (e.g., a compound listed in one of Tables 1-4), alone or in combination with one or more additional therapies (e.g., a second antibiotic). When administered to treat infectious arthritis, the dosage of the rifamycin is about 0.001 to 1000 mg/day. The compound may be given daily (e.g., a single oral dose of 2.5 to 25 mg/day) or less frequently (e.g., a single oral dose of 5, 12.5, or 25 mg/week). Treatment may be for one day to six months, nine months, one year, or longer. In one embodiment, the rifamycin is administered at an initial dose of 2.5 to 100 mg for one to seven consecutive days, followed by a maintenance dose of 0.005 to 10 mg once every one to seven days for one month, one year, or even for the life of the patient.

Neisseria gonorrhoeae is the most common bacterial cause of acute infectious arthritis in adults, spreading from infected mucosal surfaces such as the cervix, rectum, pharynx to the small joints of the hands, wrists, elbows, knees, and ankles but rarely to axial skeletal joints. Nongonococcal arthritis is usually caused by Staphylococcus aureus (45%); streptococci (9%); or gram-negative organisms, such as Enterobacter, Pseudomonas aeruginosa (40%), and Serratia marcescens (5%). Gram-negative bacterial infections tend to occur in young or elderly patients, those with severe trauma or serious underlying medical illness (e.g., renal failure or transplantation, prosthetic joints, systemic lupus erythematosus, rheumatoid arthritis diabetes, and malignancy), and IV drug users. Infections commonly begin in the urinary tract or skin. In 80% of patients, nongonococcal arthritis is monarticular (e.g., the knee, hip, shoulder, wrist, ankle, or elbow). Polyarticular bacterial arthritis usually occurs in patients with an underlying chronic arthritis (e.g., rheumatoid arthritis, osteoarthritis) or a joint prosthesis. Borrelia burgdorferi, an agent of Lyme disease, can cause acute migratory polyarthralgia with fever, headache, fatigue, and skin lesions or a more chronic intermittent monarthritis or oligoarthritis.

S. aureus and group B streptococci are the most common organisms associated with acute infectious arthritis in neonates and children over two years of age. Kingella kingae appears to be the most common cause in children under two years of age. In children, N. gonorrhoeae causes <10% of bacterial arthritis, but it is the most common cause of polyarticular infection.

Anaerobic joint infections are often mixed infections with facultative or aerobic bacteria, such as S. aureus, Staphylococcus epidermis, and Escherichia coli. The predominant anaerobic organisms are Propionibacterium acnes, Peptostreptococcus magnus, Fusobacterium spp., Clostridium spp., and Bacteroides spp. P. acnes causes infections in joints with trauma, or prior surgery. Factors predisposing to anaerobic infection include penetrating trauma, arthrocentesis, recent surgery, contiguous infection, diabetes, and malignancy.

Joint infections resulting from human bites are caused by the gram-negative organism Eikenella corrodens, group B streptococci, or oral anaerobes (e.g., Fusobacterium spp., peptostreptococci, and Bacteroides spp.). Animal bites may give rise to joint infections typically caused by S. aureus or organisms of the oral flora common to the animal. Pasteurella multocida causes half of the infections resulting from dog or cat bites. Dog and cat bites also cause infection with Pseudomonas spp., Moraxella spp., and Haemophilus spp. Rat bites cause infection with Streptobacillus moniliformis or Spirillum minus.

Joint infections in HIV-infected patients are usually caused by S. aureus, streptococci, and Salmonella. HIV-infected patients may have Reiter's syndrome, reactive arthritis, and HIV-related arthritis and arthralgias.

A subset of chronic infectious arthritis is caused in by mycobacteria such as Mycobacterium tuberculosis, Mycobacterium marinum, and Mycobacterium kansasi.

Treatment of Osteomyelitis

The invention provides methods, compositions, and kits for treating osteomyelitis (e.g., acute osteomyelitis or chronic osteomyelitis). The osteomyelitis can be treated by administering to the patient a rifamycin of any one of formulas (I)-(V) (e.g., a compound listed in one of Tables 1-4), alone or in combination with one or more additional therapies (e.g., a second antibiotic). When administered to treat osteomyelitis, the dosage of the rifamycin is about 0.001 to 1000 mg/day. The compound may be given daily (e.g., a single oral dose of 2.5 to 25 mg/day) or less frequently (e.g., a single oral dose of 5, 12.5, or 25 mg/week). Treatment may be for one day to six months, nine months, one year, or longer. In one embodiment, the rifamycin is administered at an initial dose of 2.5 to 100 mg for one to seven consecutive days, followed by a maintenance dose of 0.005 to 10 mg once every one to seven days for one month, one year, or even for the life of the patient.

Hematogenous osteomyelitis is an infection caused by bacterial seeding from the blood. Acute hematogenous osteomyelitis is characterized by an acute infection of the bone caused by the seeding of the bacteria within the bone from a remote source. Hematogenous osteomyelitis occurs primarily in children. The most common site is the rapidly growing and highly vascular metaphysis of growing bones. The apparent slowing or sludging of blood flow as the vessels make sharp angles at the distal metaphysis predisposes the vessels to thrombosis and the bone itself to localized necrosis and bacterial seeding. These changes in bone structure may be seen in x-ray images. Acute hematogenous osteomyelitis, despite its name, may have a slow clinical development and insidious onset.

Direct or contiguous inoculation osteomyelitis is caused by direct contact of the tissue and bacteria during trauma or surgery. Direct inoculation (contiguous-focus) osteomyelitis is an infection in the bone secondary to the inoculation of organisms from direct trauma, spread from a contiguous focus of infection, or sepsis after a surgical procedure. Clinical manifestations of direct inoculation osteomyelitis are more localized than those of hematogenous osteomyelitis and tend to involve multiple organisms/pathogens.

Additional categories include chronic osteomyelitis and osteomyelitis secondary to peripheral vascular disease. Chronic osteomyelitis persists or recurs, regardless of its initial cause and/or mechanism and despite aggressive intervention. Although listed as an etiology, peripheral vascular disease is actually a predisposing factor rather than a true cause of infection.

Symptoms of osteomyelitis often include high fever, fatigue, irritability and malaise. Often movement may be restricted in an infected limb or joint. Local edema, erythema, and tenderness generally accompany the infection and warmth may be present around the affected area. Sinus tract drainage may also be present at later stages of infection. Hematogenous osteomyelitis usually presents with a slow insidious progression of symptoms, while chronic osteomyelitis may include a non-healing ulcer, sinus tract drainage, chronic fatigue and malaise. Direct osteomyelitis generally presents with prominent signs and symptoms in a more localized area.

Several bacterial pathogens are commonly known to cause acute and direct osteomyelitis. For example, acute hematogenous osteomyelitis in newborns (younger than 4 months) is frequently caused by S. aureus, Enterobacter spp., and group A and B Streptococcus spp. In children aged four months to four years, acute hematogenous osteomyelitis is commonly caused by S. aureus, group A Streptococcus spp., Haemophilus influenzae, and Enterobacter spp. In children and adolescents aged 4 years to adult, acute hematogenous osteomyelitis is commonly caused by S. aureus (80%), group A Streptococcus spp., Haemophilus influenzae, and Enterobacter spp. In adults, acute hematogenous osteomyelitis is commonly caused by S. aureus and occasionally Enterobacter or Streptococcus spp.

Direct osteomyelitis is commonly caused generally by S. aureus, Enterobacter species, and Pseudomonas species. Direct osteomyelitis is frequently caused by a puncture wound through an athletic shoe. In these cases, direct osteomyelitis is commonly caused by S. aureus and Pseudomonas spp.

For patients with osteomyelitis due to trauma, the infecting agents usually include S. aureus, coliform bacilli, and Pseudomonas aeruginosa.

“Osteomyelitis” includes hematogenous osteomyelitis, direct or contiguous inoculation osteomyelitis, chronic osteomyelitis and osteomyelitis secondary to peripheral vascular disease. Osteomyelitis may be the result of infections caused by any of the above described pathogens, but also includes other pathogens having the ability to infect the bone, bone marrow, joint, or surrounding tissues.

Rifamycins

Rifamycins suitable for use in the methods, compositions, and kits of the invention are described by formulas (I)-(V) below. Methods of making these compounds are described in U.S. Patent Publication Nos. 2005-0043298, 2005-0137189, and 2005-0197333, and U.S. Provisional Application Nos. 60/638,641 and 60/732,963, each of which is hereby incorporated by reference.
Rifamycins of Formula (I)

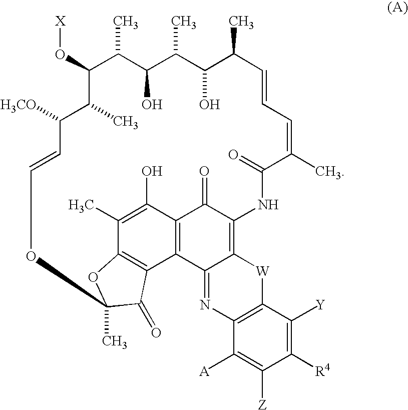

In formula (I), A is H, OH, O—(C₁-C₆ alkyl), or O—(C₁-C₄ alkaryl); W is O, S, or NR¹, where R¹ is H or C₁-C₆ alkyl; X is H or COR², where R² is C₁-C₆ alkyl which can be substituted with from 1 to 5 hydroxyl groups, or O—(C₃-C₇ alkyl), which can be substituted with from 1 to 4 hydroxyl groups; each of Y and Z is independently H, C₁-C₆ alkoxy, or Hal; and R⁴ has the following formula:

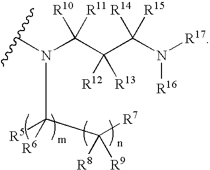

For the formula that represents R⁴, when each of m and n is 1, each of R⁵ and R⁶ is H, or R⁵ and R⁶ together are =O; R⁷ and R¹⁰ together form a single bond or a C₁-C₃ linkage, R⁷ and R¹² together form a single bond or a C₁-C₂ linkage, or R⁷ and R¹⁴ together form a single bond or a C₁ linkage; R⁸ is H, C₁-C₆ alkyl, or C₁-C₄ alkaryl, or R⁸ and R¹² together form a single bond, or R⁸ and R⁹ together are =N—OR¹⁸, where R¹⁸ is H, C₁-C₆ alkyl, or C₁-C₄ alkaryl; R⁹ is H, C₁-C₆ alkyl, or C₁-C₄ alkaryl, or R⁹ and R⁸ together are =N—OR¹⁸; R¹⁰ is H, C₁-C₆ alkyl, or C₁-C₄ alkaryl, or R¹⁰ and R¹⁷ together form a C₁-C₃ alkyl linkage, or R¹⁰ and R¹¹ together are =O; R¹¹ is H, R¹² is H, C₁-C₆ alkyl, or C₁-C₄ alkaryl; each of R¹³ and R¹⁵ is H, C₁-C₆ alkyl, or C₁-C₄ alkaryl; R¹⁴ is H, C₁-C₆ alkyl, or C₁-C₄ alkaryl; R¹⁶ is H, C₁-C₆ alkyl, C₁-C₆ alkoxy, C₆-C₁₂ aryl, heteroaryl, C₁-C₄ alkaryl, or C₁-C₄ alkheteroaryl, or R¹⁶ and R¹² together form a C₂-C₄ alkyl linkage, or R¹⁶ and R¹⁰ together form a C₁-C₂ alkyl linkage; and R¹⁷ is H, C₁-C₆ alkyl, COR¹⁹, CO₂R¹⁹, or CONHR¹⁹, CSR¹⁹, COSR¹⁹, CSOR¹⁹, CSNHR¹⁹, SO₂R¹⁹, or SO₂NHR¹⁹, where R¹⁹ is C₁-C₆ alkyl, C₆-C₁₂ aryl, C₁-C₄ alkaryl, heteroaryl, or C₁-C₄ alkheteroaryl, and where each alkyl linkage of 2 carbons or more may contain a non-vicinal O, S, or N(R²³) where R²³ is H, C₁-C₆ alkyl, COR²⁴, CO₂R²⁴, or CONHR²⁴, CSR²⁴, COSR²⁴, CSOR²⁴, CSNHR²⁴, SO₂R²⁴, or SO₂NHR²⁴, where R²⁴ is C₁-C₆ alkyl, C₆-C₁₂ aryl, C₁-C₄ alkaryl, heteroaryl, or C₁-C₄ alkheteroaryl.

When m is 0 and n is 1, R⁷ and R¹⁰ together form a single bond or a C₁-C₄ linkage, R⁷ and R¹² together form a single bond or a C₁-C₃ linkage, or R⁷ and R¹⁴ together form a single bond or a C₁-C₂ linkage; each of R⁸, R⁹, and R¹¹ is H; R¹⁵ is H, C₁-C₆ alkyl, or C₁-C₄ alkaryl; R¹⁰ is H; R¹² is H, C₁-C₆ alkyl, or C₁-C₄ alkaryl, R¹² and R¹³ together form a —CH₂CH₂— linkage, or R¹² and R¹⁶ together form a C₂-C₄ alkyl linkage; R¹³ is H, C₁-C₆ alkyl, C₁-C₄ alkaryl; R¹⁴ is H, C₁-C₆ alkyl, or C₁-C₄ alkaryl; R¹⁶ is H, C₁-C₆ alkyl, C₁-C₆ alkoxy, C₆-C₁₂ aryl, heteroaryl, C₁-C₄ alkaryl, or C₁-C₄ alkheteroaryl, or R¹⁶ and R¹² together form a C₂-C₄ alkyl linkage; and R¹⁷ is H, C₁-C₆ alkyl, COR¹⁹, CO₂R¹⁹, or CONHR¹⁹, CSR¹⁹, COSR¹⁹, CSOR¹⁹, CSNHR¹⁹, SO₂R¹⁹, or SO₂NHR¹⁹, where R¹⁹ is C₁-C₆ alkyl, C₆-C₁₂ aryl, C₁-C₄ alkaryl, heteroaryl, or C₁-C₄ alkheteroaryl, and where each alkyl linkage of 2 carbons or more may contain a non-vicinal O, S, or N(R²³) where R²³ is H, C₁-C₆ alkyl, COR²⁴, CO₂R²⁴, or CONHR²⁴, CSR²⁴, COSR²⁴, CSOR²⁴, CSNHR²⁴, SO₂R²⁴, or SO₂NHR²⁴, where R²⁴ is C₁-C₆ alkyl, C₆-C₁₂ aryl, C₁-C₄ alkaryl, heteroaryl, or C₁-C₄ alkheteroaryl.

Alternatively, for a compound of formula (I), A is OH; X is H; W, Y, and Z are as described above; and R⁴ is selected from the following groups: 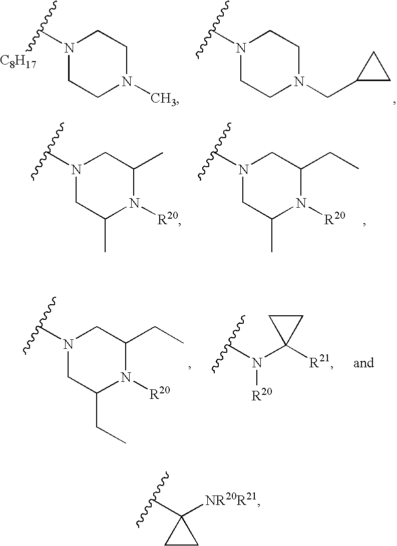
where R²¹ is H, C₁-C₆ alkyl, C₆-C₁₂ aryl, heteroaryl, C₁-C₄ alkaryl, or C₁-C₄ alkheteroaryl, R²⁰ is H, C₁-C₆ alkyl, COR¹⁹, CO₂R¹⁹, or CONHR¹⁹, CSR¹⁹, COSR¹⁹, CSOR¹⁹, CSNHR¹⁹, SO₂R¹⁹, or SO₂NHR¹⁹, where R¹⁹ is C₁-C₆ alkyl, C₆-C₁₂ aryl, C₁-C₄ alkaryl, heteroaryl, or C₁-C₄ alkheteroaryl.

Alternatively, A is OH; X is COCH₃; W, Y, and Z are as described above; and R⁴ is selected from the groups consisting of: 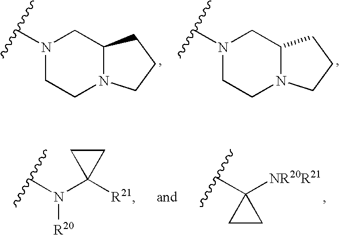
where R²¹ is H, C₁-C₆ alkyl, C₆-C₁₂ aryl, heteroaryl, C₁-C₄ alkaryl, or C₁-C₄ alkheteroaryl, R²⁰ is H, C₁-C₆ alkyl, COR¹⁹, CO₂R¹⁹, or CONHR¹⁹, CSR¹⁹, COSR¹⁹, CSOR¹⁹, CSNHR¹⁹, SO₂R¹⁹, or SO₂NHR¹⁹, where R¹⁹ is C₁-C₆ alkyl, C₆-C₁₂ aryl, C₁-C₄ alkaryl, heteroaryl, or C₁-C₄ alkheteroaryl.

Alternatively, A is H or OH; X is H or COCH₃; W, Y, and Z are as described above; and R⁴ is 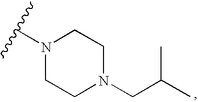
with the proviso that one or both of Y and Z are halogen.

Alternatively, A is H or OH; X is H or COCH₃; W, Y, and Z are as described above; and R⁴ is 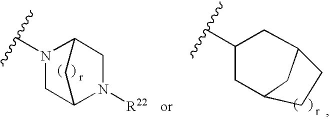
where R²² is H, C₁-C₆ alkyl, C₆-C₁₂ aryl, heteroaryl, C₁-C₄ alkaryl, C₁-C₄ alkheteroaryl, COR²⁴, CO₂R²⁴, CONHR²⁴, CSR²⁴, COSR²⁴, CSOR²⁴, CSNHR²⁴, SO₂R²⁴, or SO₂NHR ²⁴, wherein R²⁴ is C₁-C₆ alkyl, C₆-C₁₂ aryl, C₁-C₄ alkaryl, heteroaryl, or C₁-C₄ alkheteroaryl, and r is 1-2.

Alternatively, A is H or OH; X is H or COCH₃; W, Y, and Z are as described above; and R⁴ is 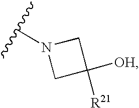
where R²¹ is H, C₁-C₆ alkyl, C₆-C₁₂ aryl, heteroaryl, C₁-C₄ alkaryl, or C₁-C₄ alkheteroaryl.

Alternatively, A is H or OH; X is H or COCH₃; W, Y, and Z are as described above; and R⁴ is 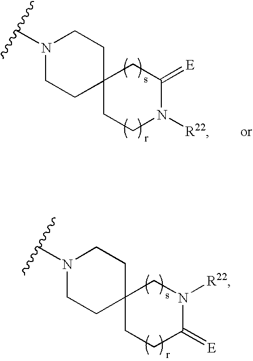
where =E is =O or (H,H), R²² is H, C₁-C₆ alkyl, C₆-C₁₂ aryl, heteroaryl, C₁-C₄ alkaryl, C₁-C₄ alkheteroaryl, COR²⁴, CO₂R²⁴, CONHR²⁴, CSR²⁴, COSR²⁴, CSOR²⁴, CSNHR²⁴, SO₂R²⁴, or SO₂NHR²⁴, where R²⁴ is C₁-C₆ alkyl, C₆-C₁₂ aryl, C₁-C₄ alkaryl, heteroaryl, or C₁-C₄ alkheteroaryl, r is 1-2, and s is 0-1.

Alternatively, A is H or OH; X is H or COCH₃; W, Y, and Z are as described above; and R⁴ is 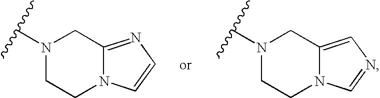
where R²² is H, C₁-C₆ alkyl, COR²⁴, CO₂R²⁴, CONHR²⁴, CSR²⁴, COSR²⁴, CSOR²⁴, CSNHR²⁴, SO₂R²⁴, or SO₂NHR²⁴, where R²⁴ is C₁-C₆ alkyl, C₆-C₁₂ aryl, C₁-C₄ alkaryl, heteroaryl, or C₁-C₄ alkheteroaryl.

In one embodiment, A is H or OH; X is H or COCH₃; W, Y, and Z are as described above; and R⁴ is 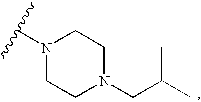
where one or both of Y and Z is F.

In another embodiment, W is O; Y is H; Z is H; A is OH, X is H or COCH₃, and R⁴ is 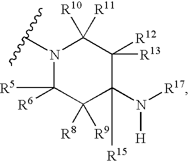
wherein each of R⁵ and R⁶ is H, or R⁵ and R⁶ together are =O, each of R⁸, R⁹, R¹², R¹³ and R¹⁵ is H, C₁-C₆ alkyl, or C₁-C₄ alkaryl, each of R¹⁰ and R¹¹ is H, C₁-C₆ alkyl, or C₁-C₄ alkaryl, or R¹⁰ and R¹¹ together are =O, R¹⁷ is H, C₁-C₆ alkyl, COR¹⁹, CO₂R¹⁹, or CONHR¹⁹, CSR¹⁹, COSR¹⁹, CSOR¹⁹, CSNHR¹⁹, SO₂R¹⁹, or SO₂NHR¹⁹, where R¹⁹ is C₁-C₆ alkyl, C₆-C₁₂ aryl, C₁-C₄ alkaryl, heteroaryl, or C₁-C₄ alkheteroaryl.

In another embodiment, W is O; Y is H; Z is H; A is H or OH, X is H or COCH₃, and R⁴ is

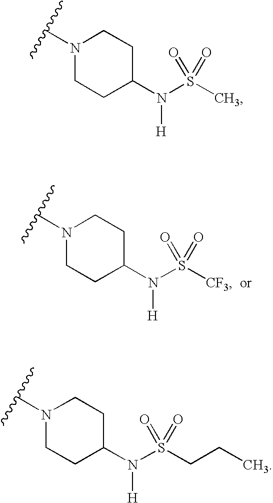

In another embodiment, W is O; Y is H; Z is H; A is H or OH, X is H or COCH₃, and R⁴ is

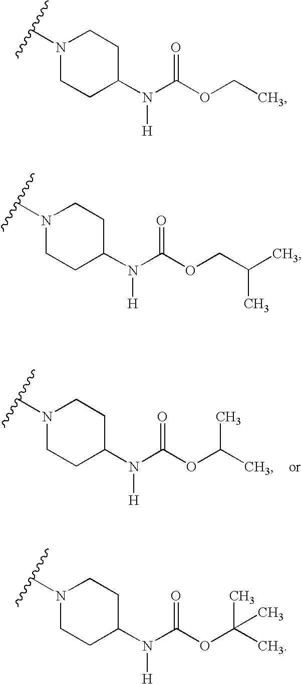

In another embodiment, W is O; Y is H; Z is H; X is H or COCH₃; A is H or OH; and R⁴ is selected from the group consisting of: 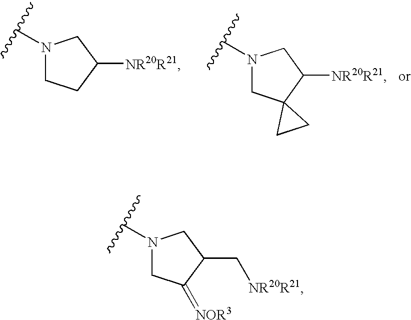
where R²⁰ and R²¹ are as described above, or

W is O; Y is H; Z is H; X is H or COCH₃, A is H or OH; and R⁴ is: 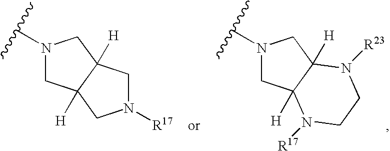
where each of R¹⁷ and R²³ is, independently, H, C₁-C₆ alkyl, COR²⁴, or CO₂R²⁴, or CONHR²⁴, where R²⁴ is C₁-C₆ alkyl, C₁-C₄ alkaryl, heteroaryl, or C₁-C₄ alkheteroaryl, or

W is O, Y is H, Z is H, X is COCH₃, A is OH, and R⁴ is selected from the group consisting of 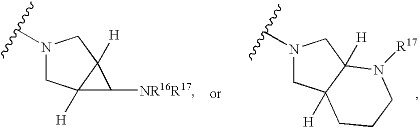
R¹⁶ and R¹⁷ are as described above.

Desirable rifamycin analogs of formula (I) include 4′-fluoro-5′-(4-isobutyl-1-piperazinyl)benzoxazinorifamycin, 4′-fluoro-5′-(1-piperazinyl)benzoxazinorifamycin, 4′-fluoro-5′-(3-methyl-1-piperazinyl)benzoxazinorifamycin, 4′-methoxy-6′-fluoro-5′-(3-methyl-1-piperazinyl)benzoxazinorifamycin, 4′,6′-difluoro-5′-[(3R,5S)-3,5-dimethyl-1-piperazinyl]benzoxazinorifamycin, 4′-fluoro-6′-methoxy-5′-[(4aS,7aS)-octahydro-6H-pyrrolo[3,4-b]pyridin-6-yl]benzoxazinorifamycin, 4′-fluoro-5′-[6-amino-3-azabicyclo[3.1.0]hex-3-yl]benzoxazinorifamycin, 25-O-deacetyl-4′-fluoro-5′-(4-isobutyl-1-piperazinyl)benzoxazinorifamycin, 25-O-deacetyl-4′-fluoro-5′-(1-piperazinyl)benzoxazinorifamycin, 25-O-deacetyl-4′-fluoro-5′-(3-methyl-1-piperazinyl)benzoxazinorifamycin, 25-O-deacetyl-4′-methoxy-6′-fluoro-5′-(3-methyl-1-piperazinyl)benzoxazinorifamycin, 25-O-deacetyl-4′,6′-difluoro-5′-[(3R,5S)-3,5-dimethyl-1-piperazinyl]benzoxazinorifamycin, 25-O-deacetyl-4′-fluoro-6′-methoxy-5′-[(4aS,7aS)-octahydro-6H-pyrrolo[3,4-b]pyridin-6-yl]benzoxazinorifamycin, 25-O-deacetyl-4′-fluoro-5′-[6-amino-3-azabicyclo[3.1.0]hex-3-yl]benzoxazinorifamycin, 25-O-deacetyl-25-(2″,3″-dihydroxypropylcarbonoxy)-5′-(4-isobutyl-1-piperazinyl)benzoxazinorifamycin, 25-O-deacetyl-25-(2″,3″-dihydroxypropylcarbonoxy)-4′-fluoro-5′-(4-isobutyl-1-piperazinyl)benzoxazinorifamycin, 25-O-deacetyl-25-(2″,3″-dihydroxypropylcarbonoxy)-4′-fluoro-5′-(1-piperazinyl)benzoxazinorifamycin, 25-O-deacetyl-25-(2″,3″-dihydroxypropylcarbonoxy)-4′-fluoro-5′-(3-methyl-1-piperazinyl)benzoxazinorifamycin, 25-O-deacetyl-25-(2″,3″-dihydroxypropylcarbonoxy)-4′-methoxy-6′-fluoro-5′-(3-methyl-1-piperazinyl)benzoxazinorifamycin, 25-O-deacetyl-25-(2″,3″-dihydroxypropylcarbonoxy)-4′,6′-difluoro-5′-[(3R,5S)-3,5-dimethyl-1-piperazinyl]benzoxazinorifamycin, 25-O-deacetyl-25-(2″,3″-dihydroxypropylcarbonoxy)-4′-fluoro-6′-methoxy-5′-[(4aS,7aS)-octahydro-6H-pyrrolo[3,4-b]pyridin-6-yl]benzoxazinorifamycin, 25-O-deacetyl-25-(2″,3″-dihydroxypropylcarbonoxy)-4′-fluoro-5′-[6-amino-3-azabicyclo[3.1.0]hex-3-yl]benzoxazinorifamycin, 4′-fluoro-5′-(4-isobutyl-1-piperazinyl)benzthiazinorifamycin, 4′-fluoro-5′-(1-piperazinyl)benzthiazinorifamycin, 4′-fluoro-5′-(3-methyl-1-piperazinyl)benzthiazinorifamycin, 4′-methoxy-6′-fluoro-5′-(3-methyl-1-piperazinyl)benzthiazinorifamycin, 4′,6′-difluoro-5′-[(3R,5S)-3,5-dimethyl-1-piperazinyl]benzthiazinorifamycin, 4′-fluoro-6′-methoxy-5′-[(4aS,7aS)-octahydro-6H-pyrrolo[3,4-b]pyridin-6-yl]benzthiazinorifamycin, 4′-fluoro-5′-[6-amino-3-azabicyclo[3.1.0]hex-3-yl]benzthiazinorifamycin, 25-O-deacetyl-4′-fluoro-5′-(4-isobutyl-1-piperazinyl)benzthiazinorifamycin, 25-O-deacetyl-4′-fluoro-5′-(1-piperazinyl)benzthiazinorifamycin, 25-O-deacetyl-4′-fluoro-5′-(3-methyl-1-piperazinyl)benzthiazinorifamycin, 25-O-deacetyl-4′-methoxy-6′-fluoro-5′-(3-methyl-1-piperazinyl)benzthiazinorifamycin, 25-O-deacetyl-4′,6′-difluoro-5′-[(3R,5S)-3,5-dimethyl-1-piperazinyl]benzthiazinorifamycin, 25-O-deacetyl-4′-fluoro-6′-methoxy-5′-[(4aS,7aS)-octahydro-6H-pyrrolo[3,4-b]pyridin-6-yl]benzthiazinorifamycin, 25-O-deacetyl-4′-fluoro-5′-[6-amino-3-azabicyclo[3.1.0]hex-3-yl]benzthiazinorifamycin, 3′-hydroxy-5′-((3R,5S)-3,5-dimethylpiperazinyl)benzoxazinorifamycin, 3′-hydroxy-5′-((3R,5S)-3,5-diethylpiperazinyl)benzoxazinorifamycin, 3′-hydroxy-5′-((3R,5S)-3-ethyl-5-methylpiperazinyl)benzoxazinorifamycin, 25-O-deacetyl-3′-hydroxy-5′-((3R,5S)-3,5-dimethylpiperazinyl)benzoxazinorifamycin, 25-O-deacetyl-3′-hydroxy-5′-((3R,5 S)-3-ethyl-5-methylpiperazinyl)benzoxazinorifamycin, 25-O-deacetyl-3′-hydroxy-5′-((3R,5S)-3,5-diethylpiperazinyl)benzoxazinorifamycin, 3′-hydroxy-5′-((4aR,7aR)octahydro-1H-pyrrolyl[3,4-b]pyridine)benzoxazinorifamycin, 3′-hydroxy-5′-((4aS,7aS)octahydro-1H-pyrrolyl[3,4-b]pyridine)benzoxazinorifamycin, 3′-hydroxy-5′-((8aR)-octahydropyrrolyl[1,2-a]pyrazine)benzoxazinorifamycin, 25-O-deacetyl-3′-hydroxy-5′-((8aR)-octahydropyrrolyl[1,2-a]pyrazine)benzoxazinorifamycin, 3′-hydroxy-5′-((8aS)-octahydropyrrolyl[1,2-a]pyrazine)benzoxazinorifamycin, 25-O-deacetyl-3′-hydroxy-5′-((8aS)-octahydropyrrolyl[1,2-a]pyrazine)benzoxazinorifamycin, 25-O-deacetyl-3′-hydroxy-5′-(4-methylpiperazinyl)benzoxazinorifamycin, 3′-hydroxy-5′-(ethyl piperidinyl-4-ylcarbamate)benzoxazinorifamycin, 25-O-deacetyl-3′-hydroxy-5′-(ethyl piperidinyl-4-ylcarbamate)benzoxazinorifamycin, 3′-hydroxy-5′-((3Z)-4-(aminomethyl)pyrrolidinyl-3-one O-methyloxime)benzoxazinorifamycin, 3′-hydroxy-5′-(5-azaspiro[2.4]heptan-7-amino-5-yl)benzoxazinorifamycin, 3′-hydroxy-5′-(5-aminopyrrolidinyl)benzoxazinorifamycin, 3′-hydroxy-5′-(4-ethylcarbamyl-1-piperidinyl)benzoxazinorifamycin, 3′-hydroxy-5′-[6-(2-trimethylsilyl)ethylcarbamyl-(1R,5S)-3-azabicyclo[3.1.0]hex-3-yl]benzoxazinorifamycin, 25-O-deacetyl-3′-hydroxy-5′-(4-ethylcarbamyl-1-piperidinyl)benzoxazinorifamycin, 3′-hydroxy-5′-[6-amino-(1R,5S)-3-azabicyclo[3.1.0]hex-3-yl]benzoxazinorifamycin, 3′-hydroxy-5′-[(4aS,7aS)-octahydro-6H-pyrrolo[3,4-b]pyridin-6-yl]benzoxazinorifamycin, 3′-hydroxy-5′-(1-piperidinyl-4-(N-phenyl)propanamide)benzoxazinorifamycin, 25-O-deacetyl-3′-hydroxy-5′-[(4aS,7aS)-octahydro-6H-pyrrolo[3,4-b]pyridin-6-yl]benzoxazinorifamycin, 25-O-deacetyl-3′-hydroxy-5′-(1-piperidinyl-4-(N-phenyl)propanamide)benzoxazinorifamycin, 3′-hydroxy-5′-(4-morpholinyl-1-piperidinyl)benzoxazinorifamycin, 3′-hydroxy-5′-(3,8-diazabicyclo[3.2.1]octan-3-yl)benzoxazinorifamycin, 25-O-deacetyl-3′-hydroxy-5′-(4-morpholinyl-1-piperidinyl)benzoxazinorifamycin, 3′-hydroxy-5′-[(4aR,7aR)-octahydro-6H-pyrrolo[3,4-b]pyridin-6-yl]benzoxazinorifamycin, 3′-hydroxy-5′-(4-(2-methylpropyl)carbamyl-1-piperidinyl)benzoxazinorifamycin, 25-O-deacetyl-3′-hydroxy-5′-(4-(2-methylpropyl)carbamyl-1-piperidinyl)benzoxazinorifamycin, 25-O-deacetyl-3′-hydroxy-5′-[(4aR,7aR)-octahydro-6H-pyrrolo[3,4-b]pyridin-6-yl]benzoxazinorifamycin, 25-O-deacetyl-3′-hydroxy-5′-(3,8-diazabicyclo[3.2.1]octan-3-yl)benzoxazinorifamycin, 3′-hydroxy-5′-(4-N,N-dimethylamino-1-piperidinyl)benzoxazinorifamycin, 25-O-deacetyl-3′-hydroxy-5′-(4-N,N-dimethylamino-1-piperidinyl)benzoxazinorifamycin, 5′-(4-ethylcarbamyl-1-piperidinyl)-N′-methylbenzodiazinorifamycin, 25-O-deacetyl-3′-hydroxy-5′-[6-amino-(1R,5S)-3-azabicyclo[3.1.0]hex-3-yl]benzoxazinorifamycin, 3′-hydroxy-5′-[6-ethylcarbamyl-(1R,5S)-3-azabicyclo[3.1.0]hex-3-yl]benzoxazinorifamycin, 3′-hydroxy-5′-[4-isopropylcarbamyl-1-piperidinyl]benzoxazinorifamycin, 3′-hydroxy-5′-[4-trifluoromethylsulfonyl-1-piperidinyl]benzoxazinorifamycin, 3′-hydroxy-5′-[4-butanamide-1-piperidinyl]benzoxazinorifamycin, 3′-hydroxy-5′-[4-methylsulfonyl-1-piperidinyl]benzoxazinorifamycin, 25-O-deacetyl-3′-hydroxy-5′-[4-propyluryl-1-piperidinyl]benzoxazinorifamycin, 25-O-deacetyl-3′-hydroxy-5′-[4-methylsulfonyl-1-piperidinyl]benzoxazinorifamycin, 3′-hydroxy-5′-[4-propyluryl-1-piperidinyl]benzoxazinorifamycin, 25-O-deacetyl-3′-hydroxy-5′-[4-isopropylcarbamyl-1-piperidinyl]benzoxazinorifamycin, 25-O-deacetyl-3′-hydroxy-5′-[4-methylcarbamyl-1-piperidinyl]benzoxazinorifamycin, 25-O-deacetyl-5′-(4-ethylcarbamyl-1-piperidinyl)-N′-methylbenzdiazinorifamycin, 3-hydroxy-5 ′-[4-methylcarbamyl-1-piperidinyl]benzoxazinorifamycin, 3-hydroxy-5′-[4-amino-1-piperidinyl]benzoxazinorifamycin, 3′-hydroxy-5′-[4-ethyluryl-1-piperidinyl]benzoxazinorifamycin, 3′-hydroxy-5′-[4-propylsulfonyl-1-piperidinyl]benzoxazinorifamycin, 25-O-deacetyl-3′-hydroxy-5′-[4-butanamide-1-piperidinyl]benzoxazinorifamycin, 25-O-deacetyl-3′-hydroxy-5′-[4-ethyluryl-1-piperidinyl]benzoxazinorifamycin, 25-O-deacetyl-3′-hydroxy-5′-[4-trifluoromethysulfonyl-1-piperidinyl]benzoxazinorifamycin, 25-O-deacetyl-3′-hydroxy-5′-[4-amino-1-piperidinyl]benzoxazinorifamycin, 3′-hydroxy-5′-[1-ethylcarbamyl-(4aR,7aR)-octahydro-6H-pyrrolo[3,4-b]pyridin-6-yl]benzoxazinorifamycin, 3′-hydroxy-5′-[1-ethylcarbamyl-(4aS,7aS)-octahydro-6H-pyrrolo[3,4-b]pyridin-6-yl]benzoxazinorifamycin, 3′-hydroxy-5′-[4-methoxyethylcarbamyl-1-piperidinyl]benzoxazinorifamycin, 25-O-deacetyl-3′-hydroxy-5′-[1-ethylcarbamyl-(4aR,7aR)-octahydro-6H-pyrrolo[3,4-b]pyridin-6-yl]benzoxazinorifamycin, 25-O-deacetyl-3′-hydroxy-5′-[1-ethylcarbamyl-(4aS,7aS)-octahydro-6H-pyrrolo[3,4-b]pyridin-6-yl]benzoxazinorifamycin, 25-O-deacetyl-3′-hydroxy-5′-[4-acetamide-1-piperidinyl]benzoxazinorifamycin, 3′-hydroxy-5′-[4-acetyl-1-piperidinyl]benzoxazinorifamycin, 3′-hydroxy-5′-[4-S-methylthiocarbamyl-1-piperidinyl]benzoxazinorifamycin, 25-O-deacetyl-3′-hydroxy-5′-[1-acetyl-(4aR,7aR)-octahydro-6H-pyrrolo[3,4-b]pyridin-6-yl]benzoxazinorifamycin, 25-O-deacetyl-3′-hydroxy-5′-[1-acetyl-(4aS,7aS)-octahydro-6H-pyrrolo[3,4-b]pyridin-6-yl]benzoxazinorifamycin, 3′-hydroxy-5′-[1-acetyl-(4aR,7aR)-octahydro-6H-pyrrolo[3,4-b]pyridin-6-yl]benzoxazinorifamycin, 3′-hydroxy-5′-[1-acetyl-(4aS,7aS)-octahydro-6H-pyrrolo[3,4-b]pyridin-6-yl]benzoxazinorifamycin, 3′-hydroxy-5′-[4-(2,2-dimethylethyl)carbamyl-1-piperidinyl]benzoxazinorifamycin, 3′-hydroxy-5′-[4-(4-(S-methylthiocarbamyl)-1-piperidinylcarbonyl)amino-1-piperidinyl]benzoxazinorifamycin, 3′-hydroxy-5′-[4-(4-methylpiperazinylcarbonyl)amino-1-piperidinyl]benzoxazinorifamycin, 3′-hydroxy-5′-[4-ethylcarbamylmethyl-1-piperidinyl]benzoxazinorifamycin, 25-O-deacetyl-3′-hydroxy-5′-[4-(2,2-dimethylethyl)carbamyl-1-piperidinyl]benzoxazinorifamycin, 3′-hydroxy-5′-[6-N,N-dimethylamino-(1R,5S)-3-azabicyclo[3.1.0]hex-3-yl]benzoxazinorifamycin, 3′-hydroxy-5′-[6-N,N-dimethylamino-(1R,5s)-3-azabicyclo[3.1.0]hex-3-yl]benzoxazinorifamycin, 3′-hydroxy-5′-[4-acetylaminomethyl-1-piperidinyl]benzoxazinorifamycin, 25-O-deacetyl-3′-hydroxy-5′-[4-acetylaminomethyl-1-piperidinyl]benzoxazinorifamycin, 3′-hydroxy-5′-[4-phenyl-1-piperidinyl]benzoxazinorifamycin, 3′-hydroxy-5′-[1-methyl-(4aS,7aS)-octahydro-6H-pyrrolo[3,4-b]pyridin-6-yl]benzoxazinorifamycin, 3′-hydroxy-5′-[1-methyl-(4aR,7aR)-octahydro-6H-pyrrolo[3,4-b]pyridin-6-yl]benzoxazinorifamycin, 25-O-deacetyl-3′-hydroxy-5′-[1-methyl-(4aS,7aS)-octahydro-6H-pyrrolo[3,4-b]pyridin-6-yl]benzoxazinorifamycin, 25-O-deacetyl-3′-hydroxy-5′-[1-methyl-(4aR,7aR)-octahydro-6H-pyrrolo[3,4-b]pyridin-6-yl]benzoxazinorifamycin, 25-O-deacetyl-3′-hydroxy-5′-[4-ethylcarbamylmethyl-1-piperidinyl]benzoxazinorifamycin, 3′-hydroxy-5′-[4-(2-hydroxyethyl)-1-piperidinyl]benzoxazinorifamycin, 25-O-deacetyl-3′-hydroxy-5′-[4-phenyl-1-piperidinyl]benzoxazinorifamycin, 25-O-deacetyl-3′-hydroxy-5′-[4-methoxyethylcarbamyl-1-piperidinyl]benzoxazinorifamycin, 5′-[(3R,5S)-3,5-dimethyl-1-piperazinyl]benzthiazinorifamycin, 5′-[(3S,5R)-3,5-dimethyl-1-piperazinyl]benzthiazinorifamycin, 25-O-deacetyl-5′-[(3R,5S)-3,5-dimethyl-1-piperazinyl]benzthiazinorifamycin, 25-O-deacetyl-5′-[(3S,5R)-3,5-dimethyl-1-piperazinyl]benzthiazinorifamycin, 25-O-deacetyl-3′-hydroxy-5′-[4-(2-hydroxyethyl)-1-piperidinyl]benzoxazinorifamycin, 25-O-deacetyl-3′-hydroxy-5′-[4-propylsulfonyl-1-piperidinyl]benzoxazinorifamycin, 5′-[(2S,5R)-4-(cyclopropylmethyl)-2,5-dimethylpiperazinyl]benzthiazinorifamycin, 5′-[(2R,5S)-4-(cyclopropylmethyl)-2,5-dimethylpiperazinyl]benzthiazinorifamycin, 5′-[4-N,N-dimethylamino-1-piperidinyl]benzthiazinorifamycin, 25-O-deacetyl-5′-[(2S,5R)-4-(cyclopropylmethyl)-2,5-dimethylpiperazinyl]benzthiazinorifamycin, 25-O-deacetyl-5′-[(2R,5S)-4-(cyclopropylmethyl)-2,5-dimethylpiperazinyl]benzthiazinorifamycin, 3′-hydroxy-5′-[4-methyl-4-N,N-dimethylamino-1-piperidinyl]benzoxazinorifamycin, 3′-hydroxy-5′-[4-methyl-4-acetylamino-1-piperidinyl]benzoxazinorifamycin, 25-O-deacetyl-3′-hydroxy-5′-[4-methyl-4-N,N-dimethylamino-1-piperidinyl]benzoxazinorifamycin, 25-O-deacetyl-3′-hydroxy-5′-[4-methyl-4-acetylamino-1-piperidinyl]benzoxazinorifamycin, 3′-hydroxy-5′-[(3R)-N,N-dimethylamino-1-pyrrolidinyl]benzoxazinorifamycin, 3′-hydroxy-5′-[(3S)-N,N-dimethylamino-1-pyrrolidinyl]benzoxazinorifamycin, 5′-[(8aS)octahydropyrrolo[1,2-a]pyrazin-2-yl]benzthiazinorifamycin, 5′-[(8aR)octahydropyrrolo[1,2-a]pyrazin-2-yl]benzthiazinorifamycin, 25-O-deacetyl-5′-[(8aS)octahydropyrrolo[1,2-a]pyrazin-2-yl]benzthiazinorifamycin, 25-O-deacetyl-5′-[(8aR)octahydropyrrolo[1,2-a]pyrazin-2-yl]benzthiazinorifamycin, or 25-O-deacetyl-3′-hydroxy-5′-[3-hydroxy-1-azetidinyl]benzoxazinorifamycin.

Rifamycins of Formula (II)

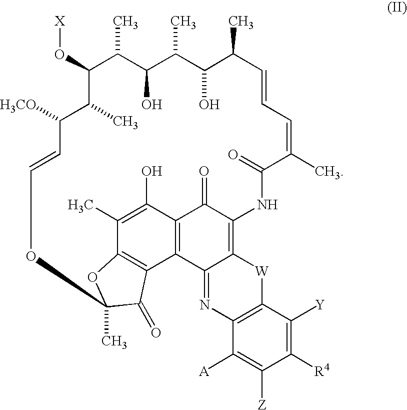

In formula (II), A is H, OH, O—(C₁-C₆ alkyl), O—(C₁-C₄ alkaryl), O—(C₆-C₁₂ aryl), O—(C₁-C₉ heteroaryl), or O—(C₁-C₄ alkheteroaryl). Preferably A is H, OH, O—(C₁-C₆ alkyl), or O—(C₁-C₄ alkaryl).

W is O, S, or NR¹, where R¹ is H, C₁-C₆ alkyl, C₁-C₄ alkaryl, or C₁-C₄ alkheteroaryl. Preferably R¹ is H or C₁-C₆ alkyl.

X is H or COR², where R² is C₁-C₆ alkyl, which can be substituted with from 1 to 5 OH groups, or O—(C₃-C₇ alkyl), which can be substituted with from 1 to 4 OH groups, with each carbon atom of the alkyl group bonded to no more than one oxygen. R² can also represent C₆-C₁₂ aryl, C₁-C₄ alkaryl, C₁-C₉ heteroaryl, or C₁-C₄ alkheteroaryl.

R⁴ is OR⁵, SR⁵, or NR⁵R⁶, where R⁵ and R⁷, which is a substituent on Z as described below, together represent a bond or form a substituted or unsubstituted C₁-C₄ linkage (i.e., the R⁴ and Z substituents form a ring) and R⁶ is H, C₁-C₆ alkyl, C₁-C₆ alkaryl, COR⁹, CO₂R⁹, CONHR⁹, CSR⁹, COSR⁹, CSOR⁹, CSNHR⁹, SO₂R⁹, or SO₂NHR⁹, where R⁹ is C₁-C₆ alkyl, C₆-C₁₂ aryl, C₁-C₄ alkaryl, heteroaryl, or C₁-C₄ alkheteroaryl. R⁶ can also represent C₆-C₁₂ aryl, C₁-C₉ heteroaryl, or C₁-C₄ alkheteroaryl.

Y is H, Hal, or OR³, where R³ is C₁-C₆ alkyl, C₆-C₁₂ aryl, C₁-C₄ alkaryl, C₁-C₉ heteroaryl, or C₁-C₄ alkheteroaryl. Preferably, R³ is C₁-C₆ alkyl or C₁-C₄ alkaryl.

Z is (CR¹¹R¹²)ₙNR⁷R⁸, where n is 0 or 1, R⁸ is H, C₁-C₆ alkyl, C₁-C₄ alkaryl, COR¹⁰, CO₂R¹⁰, CONHR¹⁰, CSR¹⁰, COSR¹⁰, CSOR¹⁰, CSNHR¹⁰, SO₂R¹⁰, or SO₂NHR¹⁰, where R¹⁰ is C₁-C₆ alkyl, C₆-C₁₂ aryl, C₁-C₄ alkaryl, heteroaryl, or C₁-C₄ alkheteroaryl. R⁸ can also represent C₆-C₁₂ aryl, C₁-C₉ heteroaryl, or C₁-C₄ alkheteroaryl, or R⁸ does not exist and a double bond is formed between N and an R⁵-R⁷ C₁carbon linkage. Each of R¹¹ and R¹² is, independently, H, C₁-C₆ alkyl, C₁-C₄ alkaryl, or C₁-C₄ alkheteroaryl, or R¹² does not exist and a double bond is formed between N and the carbon bearing R¹¹.

Alternatively, for a compound of formula (II), each of A, W, X is, respectively, as defined above; Z is H, Hal, or OR³, where R³ is as previously defined; R⁴ is OR⁵, SR⁵, or NR⁵R⁶, where R⁶ is as previously defined and R⁵, together with R⁷, which is a substituent on Y as described below, represent a bond or form a substituted or unsubstituted C₁-C₄ linkage (i.e., the R⁴ and Y substituents form a ring); and Y is (CR¹¹R¹²)ₙNR⁷R⁸, where each of n and R⁸ is as previously defined.

In one embodiment, W is O, S, or NR¹, where R¹ is H or C₁-C₆ alkyl. In another embodiment, X can be either H or COR², where R² is C₁-C₆ alkyl, which can be substituted with from 1 to 5 OH groups, or O—(C₃-C₇ alkyl), which can be substituted with from 1 to 4 OH groups, with each carbon atom of the alkyl group bonded to no more than one oxygen. In yet another embodiment, A is OH.

Desirable compounds include the following compounds of formula (II):

(a) the compound where A is OH, X is COCH₃, W is O, Z is H, and, together, Y and R⁴ are:

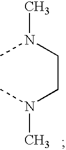

(b) the compound where A is OH, X is H, W is O, Z is H, and, together, Y and R⁴ are:

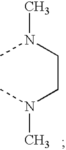

(c) the compound where A is OH, X is COCH₃, W is O, Z is H, and, together, Y and R⁴ are:

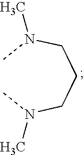

(d) the compound where A is OH, X is H, W is O, Z is H, and, together, Y and R⁴ are

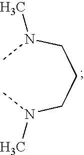

(e) the compound where A is OH, X is COCH₃, W is O, Z is H, and, together, Y and R⁴ are:

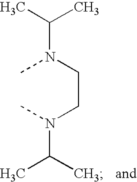

(f) the compound where A is OH, X is COCH₃, W is O, Z is H, and, together, Y and R⁴ are:

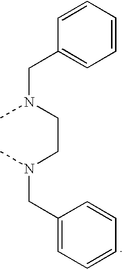

Rifamycins of Formula (III)

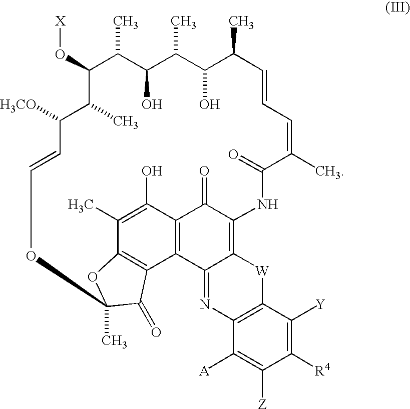

In formula (III), A is H, OH, O—(C₁₋₆ alkyl), O—(C₁₋₄ alkaryl), O—(C₆₋₁₂ aryl), O—(C₁₋₉ heteroaryl), or O—(C₁₋₄ alkheteroaryl); W is O, S, or NR¹, wherein R¹ is H, C₁₋₆ alkyl, C₁₋₄ alkaryl, or C₁₋₄ alkheteroaryl; X is H or COR², wherein R² is C₁₋₆ alkyl, which can be substituted with 1-5 OH groups, O—(C₃₋₇ alkyl), which can be substituted with 1-4 OH groups, C₆₋₁₂ aryl, C₁₋₄ alkaryl, C₁₋₉ heteroaryl, or C₁₋₄ alkheteroaryl, wherein each alkyl carbon is bonded to no more than one oxygen atom; Y is H, Hal, or OR^{Y3}, wherein R^{Y3} is C₁₋₆ alkyl, C₆₋₁₂ aryl, C₁₋₄ alkaryl, C₁₋₉ heteroaryl, or C₁₋₄ alkheteroaryl; Z is H, Hal, or OR ^{Z3}, wherein R^{Z3} is C₁₋₆ alkyl, C₆₋₁₂ aryl, C₁₋₄ alkaryl, C₁₋₉ heteroaryl, or C₁₋₄ alkheteroaryl; and R⁴ has the formula: 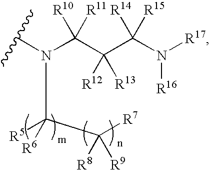
wherein, when each of m and n is 1 in the R⁴ substituent: each of R⁵ and R⁶ is H, or R⁵ and R⁶ together are =O; R⁷ and R¹⁰ together form a single bond or a C₁₋₃ linkage, which optionally contains a non-vicinal O, S, or N(R²³), R⁷ and R¹² together form a single bond or a C₁₋₂ linkage, which optionally contains a non-vicinal O, S, or N(R²³), R⁷ and R¹⁴ together form a single bond or a C₁ linkage, or R⁷ and R¹⁶ together form a single bond or a C₁ linkage, where R²³ is H, C₁₋₆ alkyl, C₁₋₄ alkaryl, C₁₋₄ alkheteroaryl, COR^{24b}, CO₂R^{24a}, CONR^{24a}R^{24b}, CSR^{24b}, COSR^{24a}, CSOR^{24a}, CSNR^{24a}R^{24b}, SO₂R^{24a}, or SO₂NR^{24a}R^{24b}, wherein R^{24a} is C₁₋₆ alkyl, C₆₋₁₂ aryl, C₁₋₄ alkaryl, C₁₋₉ heteroaryl, or C₁₋₄ alkheteroaryl, R^{24b} is H, C₁₋₆ alkyl, C₆₋₁₂ aryl, C₁₋₄ alkaryl, C₁₋₉ heteroaryl, or C₁₋₄ alkheteroaryl, or R^{24a} and R^{24b} together form a C₂₋ linkage, optionally containing a non-vicinal O; R⁸ is H, C₁₋₆ alkyl, C₁₋₄ alkaryl, C₁₋₄ alkheteroaryl, R⁸ and R⁹ together are =O or =N—OR¹⁸, where R¹⁸ is H, C₁₋₆ alkyl, C₁₋₄ alkaryl, or C₁₋₄ alkheteroaryl, or R⁸ and R¹² together form a single bond; R⁹ is H, C₁₋₆ alkyl, C₁₋₄ alkaryl, C₁₋₄ alkheteroaryl, or R⁹ and R⁸ together are =O or =N—OR ¹⁸, where R¹⁸ is as previously defined; R¹⁰ is H, C₁₋₆ alkyl, C₁₋₄ alkaryl, C₁₋₄ alkheteroaryl, R¹⁰ and R⁷ together form a ring as previously defined, R¹⁰ and R¹¹ together are =O, R¹⁰ and R¹⁶ together form a C₁₋₂ alkyl linkage, which optionally contains a non-vicinal O, S, or N(R²³), or R¹⁰ and R¹⁷ together form a C₁₋₃ alkyl linkage, which optionally contains a non-vicinal O, S, or N(R²³), where R²³ is as previously defined; R¹¹ is H; R¹² is H, C₁₋₆ alkyl, C₁₋₄ alkaryl, C₁₋₄ alkheteroaryl, R¹² and R¹⁶ together form a C₂₋₄ alkyl linkage, which optionally contains a non-vicinal O, S, or N(R²³), or R¹² and R⁷ or R¹² and R⁸ together form a ring as previously defined; R¹³ is H, C₁₋₆ alkyl, C₁₋₄ alkaryl, or C₁₋₄ alkheteroaryl; R¹⁴ is H, C₁₋₆ alkyl, C₁₋₄ alkaryl, C₁₋₄ alkheteroaryl, or R¹⁴ and R⁷ together form a ring as previously defined; R¹⁵ is H, C₁₋₆ alkyl, C₁₋₄ alkaryl, or C₁₋₄ alkheteroaryl; R¹⁶ is H, C₁₋₆ alkyl, C₁₋₆ alkoxy, C₆₋₁₂ aryl, C₁₋₉ heteroaryl, C₁₋₄ alkaryl, C₁₋₄ alkheteroaryl, or R¹⁶ and R⁷, R¹⁶ and R¹⁰, or R¹⁶ and R¹² together form rings as previously defined; and R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl, or R¹⁷ and R¹⁰ together form a ring as previously defined.

In one embodiment, W is O; Y is H; Z is H; A is OH, X is H or COCH₃, and R⁴ is: 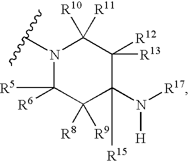
wherein each of R⁵ and R⁶ is H, or R⁵ and R⁶ together are =O, each of R⁸, R⁹, R¹², R¹³ and R¹⁵ is H, C₁₋₆ alkyl, or C₁₋₄ alkaryl, each of R¹⁰ and R¹¹ is H, C₁₋₆ alkyl, or C₁₋₄ alkaryl, or R¹⁰ and R¹¹ together are =O, R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl.

In another embodiment, W is O; Y is H; Z is H; A is H or OH, X is H or COCH₃, and R⁴ is:

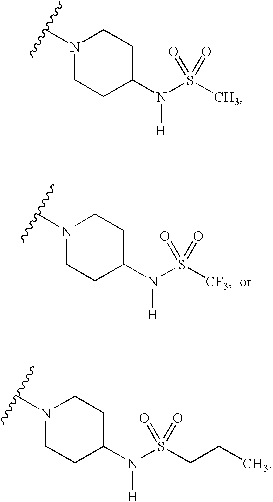

In another embodiment, W is O; Y is H; Z is H; A is H or OH, X is H or COCH₃, and R⁴ is:

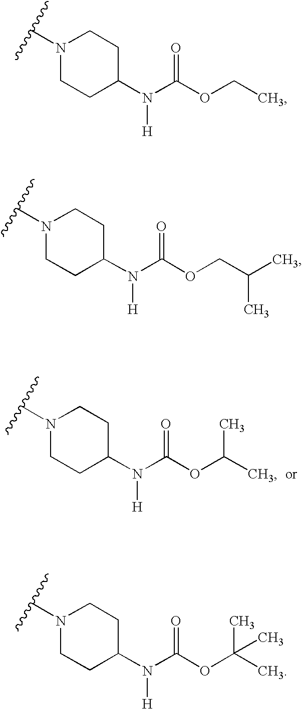

In yet another embodiment, W is O; Y is H; Z is H; X is H or COCH₃, A is H or OH; and R⁴ is: 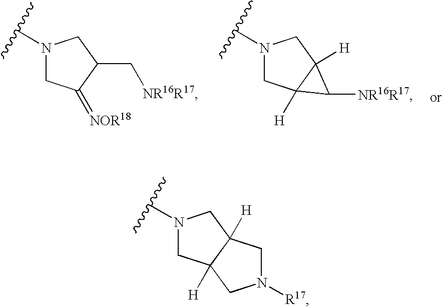
where R¹⁶ is H, C₁₋₆ alkyl, C₁₋₆ alkoxy, C₆₋₁₂ aryl, C₁₋₉ heteroaryl, C₁₋₄ alkaryl, or C₁₋₄ alkheteroaryl; R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl; and R¹⁸ is H, C₁₋₆ alkyl, C₁₋₄ alkaryl, or C₁₋₄ alkheteroaryl.

Alternatively, for a compound of formula (III), when m is 0 and n is 1 in the formula that represents R⁴: R⁷ and R¹⁰ together form a single bond or a C₁₋₄ linkage, which optionally contains a non-vicinal O, S, or N(R²³), R⁷ and R¹² together form a single bond or a C₁₋₃ linkage, which optionally contains a non-vicinal o, S, or N(R²³), or R⁷ and R¹⁴ together form a single bond or a C₁₋₂ linkage, which optionally contains a non-vicinal O, S, or N(R²³), where R²³ is as previously defined; each of R⁸ and R⁹ is H; R¹⁰ is H or R¹⁰ and R⁷ together form a single bond or a C₁₋₄ linkage, which optionally contains a non-vicinal O, S, or N(R²³), where R²³ is as previously defined; R¹¹ is H; R¹² is H, C₁₋₆ alkyl, C₁₋₄ alkaryl, C₁₋₄ alkheteroaryl, R¹² and R⁷ together form a single bond or a C₁₋₃ linkage, which optionally contains a non-vicinal O, S, or N(R²³), R¹² and R¹³ together form a —CH₂CH₂— linkage, or R¹² and R¹⁶ together form a C₂₋₄ alkyl linkage, which optionally contains a non-vicinal O, S, or N(R²³), where R²³ is as previously defined; R¹³ is H, C₁₋₆ alkyl, C₁₋₄ alkaryl, C₁₋₄ alkheteroaryl, or R¹³ and R¹² together form a —CH₂CH₂— linkage; R¹⁴ is H, C₁₋₆ alkyl, C₁₋₄ alkaryl, C₁₋₄ alkheteroaryl, or R¹⁴ and R⁷ together form a single bond or a C₁₋₂ linkage, which optionally contains a non-vicinal O, S, or N(R²³), where R²³ is as previously defined; R¹⁵ is H, C₁₋₆ alkyl, C₁₋₄ alkaryl, or C₁₋₄ alkheteroaryl; R¹⁶ is H, C₁₋₆ alkyl, C₁₋₆ alkoxy, C₆₋₁₂ aryl, C₁₋₉ heteroaryl, C₁₋₄ alkaryl, C₁₋₄ alkheteroaryl, or R¹⁶ and R¹² together form a C₂₋₄ alkyl linkage, which optionally contains a non-vicinal O, S, or N(R²³), where R²³ is as previously defined; and R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is as previously defined and where each alkyl linkage of 2 carbons or more may contain a non-vicinal O, S, or N(R²³) where R²³ is as previously defined.

In one embodiment, W is O; Y is H; Z is H; X is H or COCH₃; A is H or OH; and R⁴ is selected from the group consisting of: 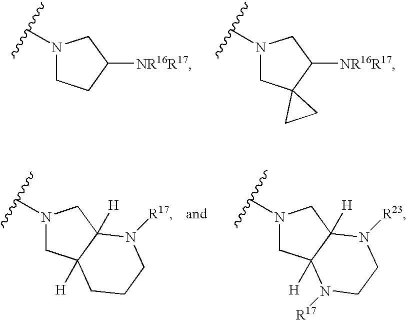
where R¹⁶ is H, C₁₋₆ alkyl, C₁₋₆ alkoxy, C₆₋₁₂ aryl, C₁₋₉ heteroaryl, C₁₋₄ alkaryl, or C₁₋₄ alkheteroaryl, and each of R¹⁷ and R²³ is as previously defined.

Alternatively, for a compound of formula (III), A is OH; X is H; W, Y, and Z are as described above; and R⁴ is selected from the group consisting of: 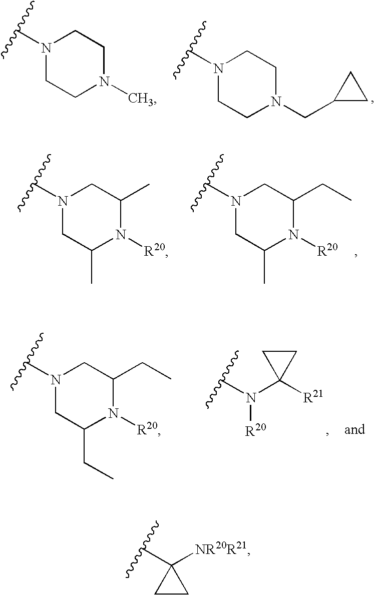
where R²¹ is H, C₁₋₆ alkyl, C₆₋₁₂ aryl, C₁₋₉ heteroaryl, C₁₋₄ alkaryl, or C₁₋₄ alkheteroaryl, R²⁰ is H, C₁₋₆ alkyl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl.

Alternatively, A is OH; X is COCH₃; W, Y, and Z are as defined above; and R⁴ is selected from the groups consisting of: 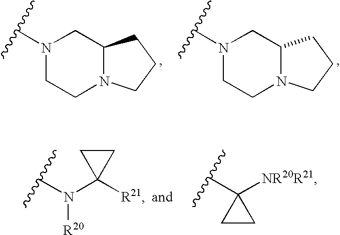
where R²¹ is H, C₁₋₆ alkyl, C₆₋₁₂ aryl, C₁₋₉ heteroaryl, C₁₋₄ alkaryl, or C₁₋₄ alkheteroaryl, R²⁰ is H, C₁₋₆ alkyl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl.

Alternatively, A is H or OH; X is H or COCH₃; W, Y, and Z are as defined above; and R⁴ is: 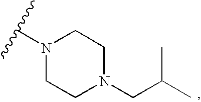
with the proviso that one or both of Y and Z are halogen. In one embodiment, one or both of Y and Z is F.

Alternatively, A is H or OH; X is H or COCH₃; W, Y, and Z are as defined above; and R⁴ is: 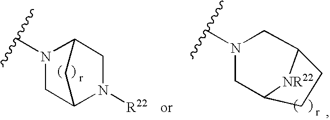
where R²² is H, C₁₋₆ alkyl, C₆₋₁₂ aryl, C₁₋₉ heteroaryl, C₁₋₄ alkaryl, C₁₋₄ alkheteroaryl, COR²⁴, CO₂R²⁴, CONHR²⁴, CSR²⁴, COSR²⁴, CSOR²⁴, CSNHR²⁴, SO₂R²⁴, or SO₂NHR²⁴, wherein R²⁴ is C₁₋₆ alkyl, C₆₋₁₂ aryl, C₁₋₄ alkaryl, C₁₋₉ heteroaryl, or C₁₋₄ alkheteroaryl, and r is 1-2.

Alternatively, A is H or OH; X is H or COCH₃; W, Y, and Z are as defined above; and R⁴ is: 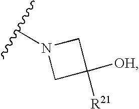
where R²¹ is H, C₁₋₆ alkyl, C₆₋₁₂ aryl, C₁₋₉ heteroaryl, C₂₋₉ heterocyclyl, C₁₋₄ alkaryl, or C₁₋₄ alkheteroaryl.

Alternatively, A is H or OH; X is H or COCH₃; W, Y, and Z are as defined above; and R⁴ is: 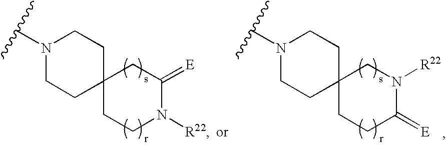
where =E is =O or (H,H), R²² is H, C₁₋₆ alkyl, C₆₋₁₂ aryl, C₁₋₉ heteroaryl, C₁₋₄ alkaryl, C₁₋₄ alkheteroaryl, COR²⁴, CO₂R²⁴, CONHR²⁴, CSR²⁴, COSR²⁴, CSOR²⁴, CSNHR²⁴, SO₂R²⁴, or SO₂NHR²⁴, where R²⁴ is C₁₋₆ alkyl, C₆₋₁₂ aryl, C₁₋₄ alkaryl, C₁₋₉ heteroaryl, or C₁₋₄ alkheteroaryl, r is 1-2, and s is 0-1.

Alternatively, A is H or OH; X is H or COCH₃; W, Y, and Z are as defined above; and R⁴ is:

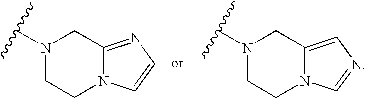

Other compounds of formula (III) are provided below. 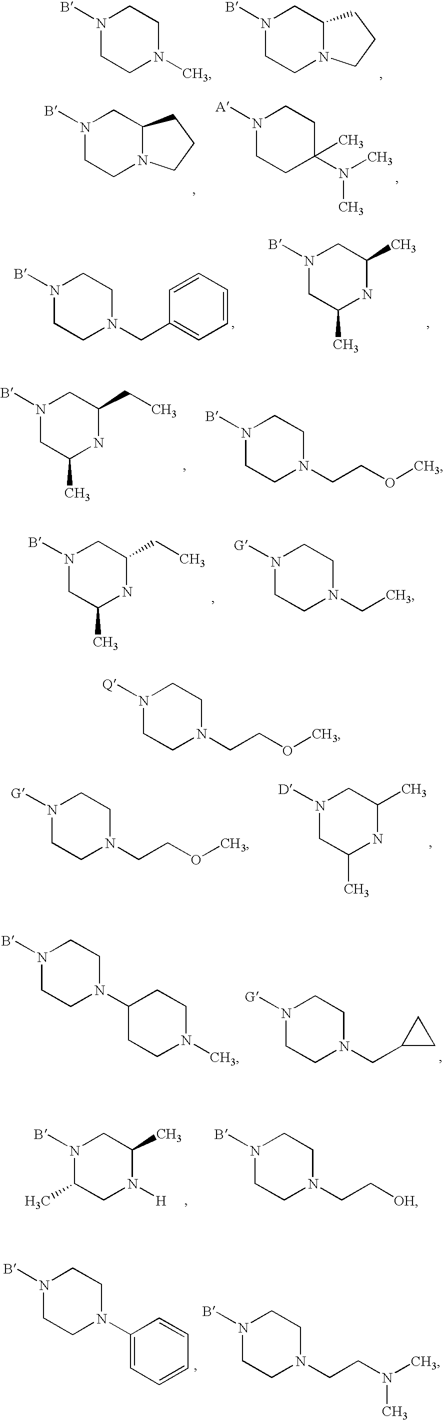 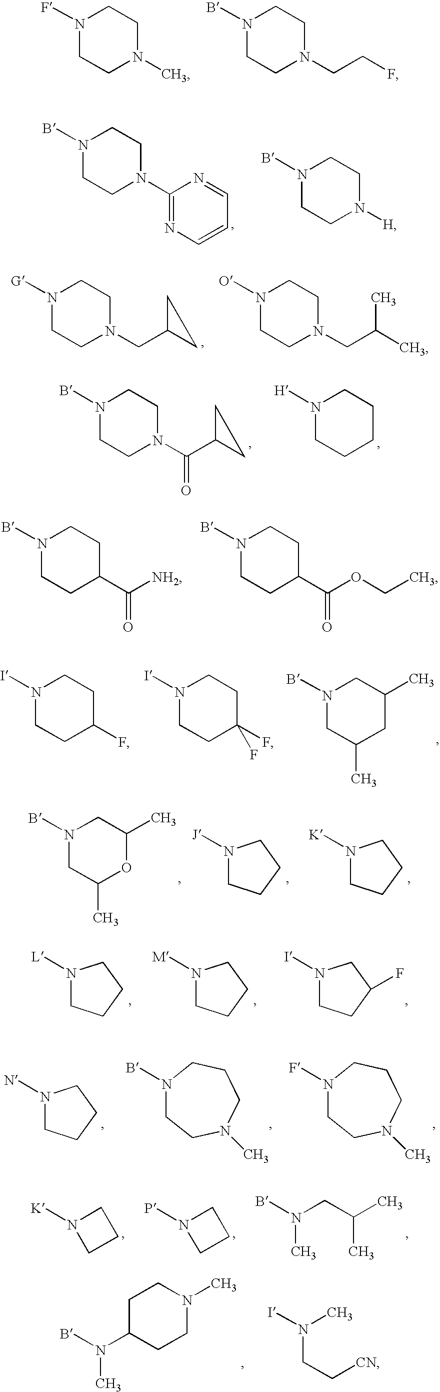 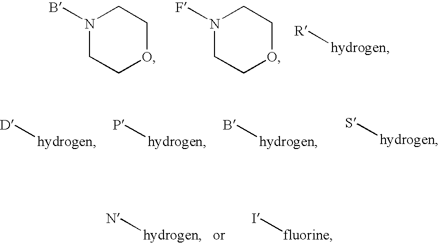
wherein A′ is 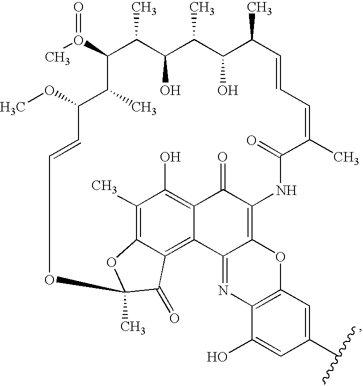
B′ is 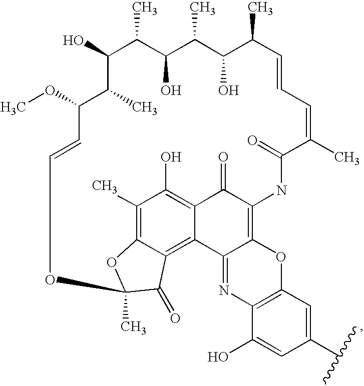
C′ is 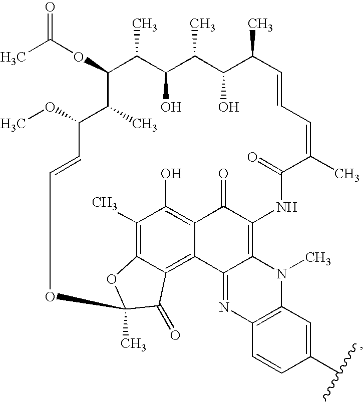
D′ is 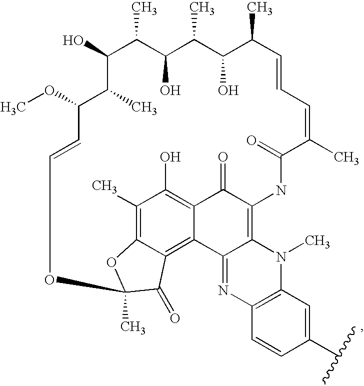
E′ is 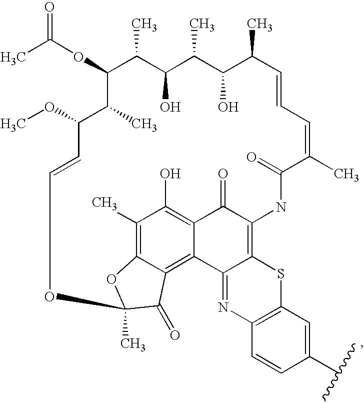
F′ is 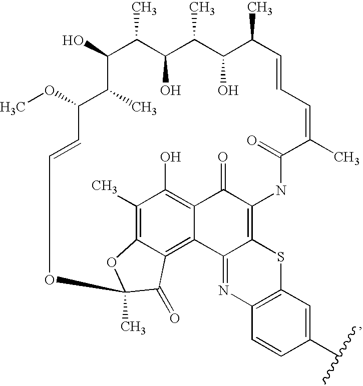
G′ is 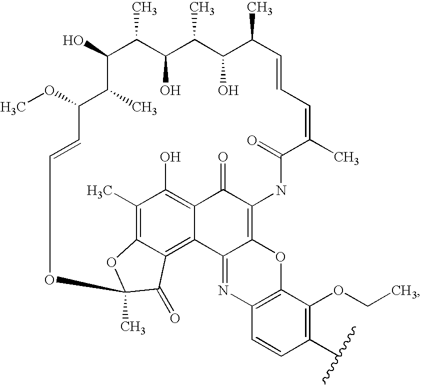
H′ is 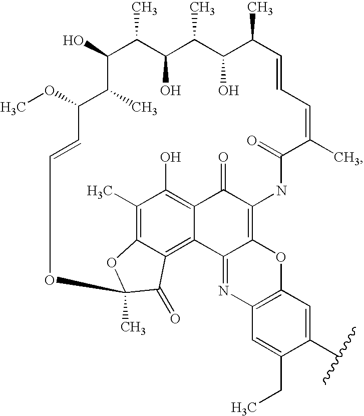
I′ is 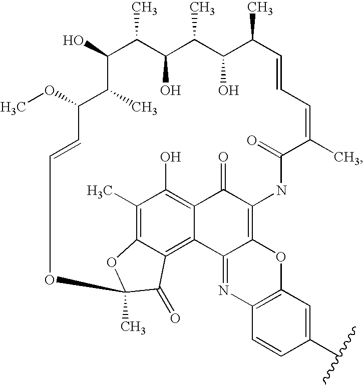
J′ is 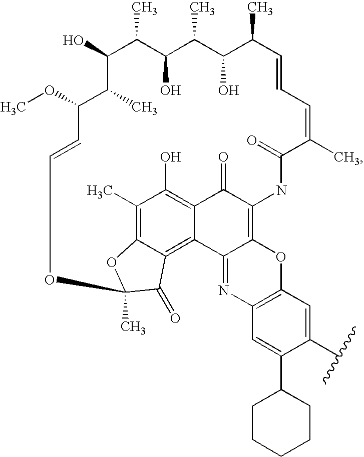
K′ is 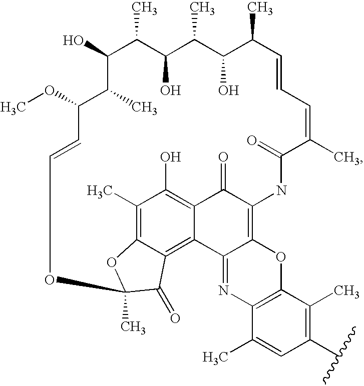
L′ is 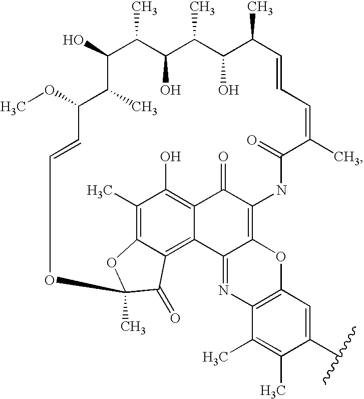
M′ is 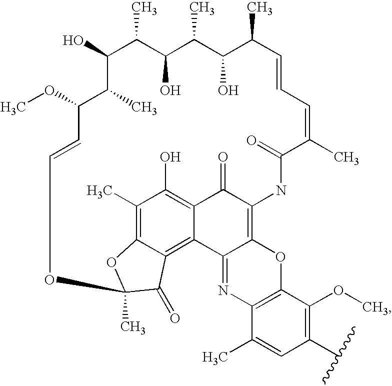
N′ is 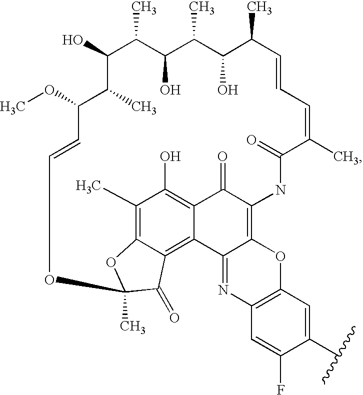
O′ is 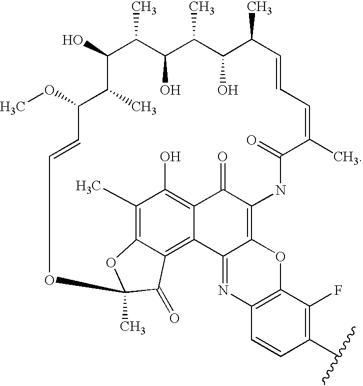
P′ is 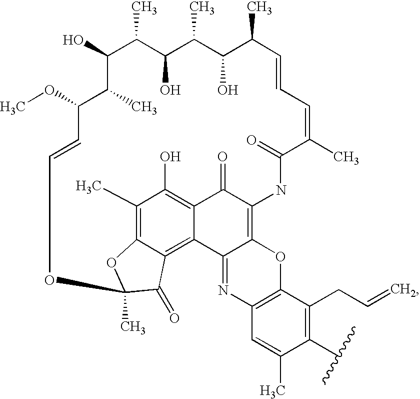
Q′ is 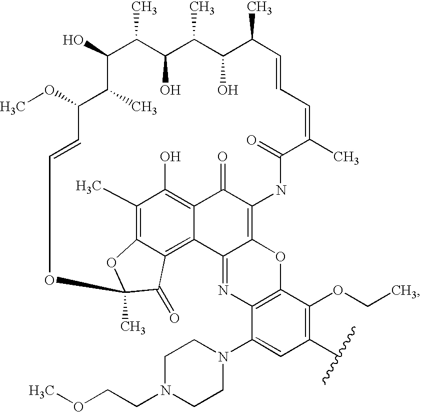
R′ is 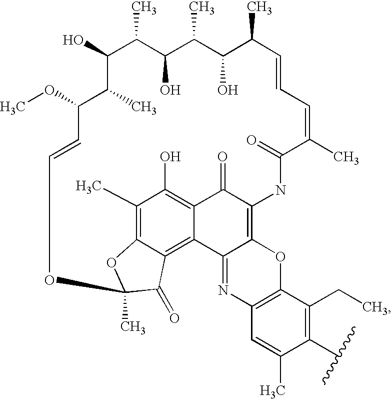
and S′ is

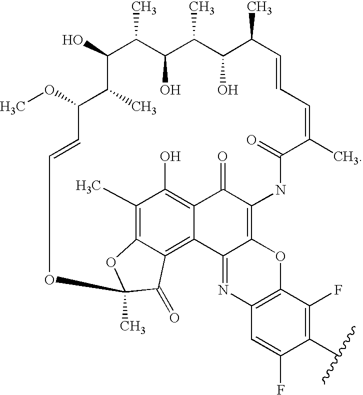

Rifamycins of Formula (IV)

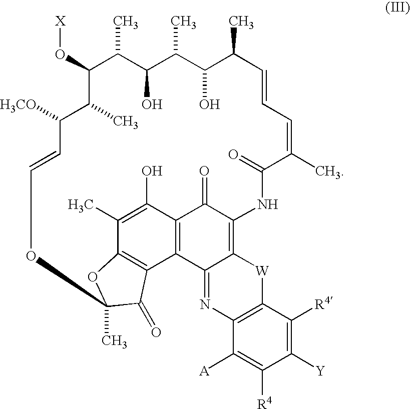

In formula (IV), A is H, OH, O—(C₁₋₆ alkyl), O—(C₁₋₄ alkaryl), O—(C₃₋₁₂ aryl), O—(C₁₋₉ heteroaryl), or O—(C₁₋₄ alkheteroaryl); W is O, S, or NR¹, wherein R¹ is H, C₁₋₆ alkyl, C₁₋₄ alkaryl, or C₁₋₄ alkheteroaryl; X is H or COR², wherein R² is C₁₋₆ alkyl, which can be substituted with 1-5 OH groups, O—(C₃₋₇ alkyl), which can be substituted with 1-4 OH groups, C₆₋₁₂ aryl, C₁₋₄ alkaryl, C₁₋₉ heteroaryl, or C₁₋₄ alkheteroaryl, wherein each alkyl carbon is bonded to no more than one oxygen atom; Y is H, Hal, or OR^{Y3}, wherein R^{Y3} is C₁₋₆ alkyl, C₆₋₁₂ aryl, C₁₋₄ alkaryl, C₁₋₉ heteroaryl, or C₁₋₄ alkheteroaryl; and each of R⁴ and R^{4′}, independently, is H or has the formula:

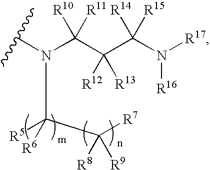

where R⁴ and R^{4′} cannot both be H at the same time.

When each of m and n is 1: each of R⁵ and R⁶ is H, or R⁵ and R⁶ together are =O; R⁷ and R¹⁰ together form a single bond or a C₁₋₃ linkage, which optionally contains a non-vicinal O, S, or N(R²³), R⁷ and R¹² together form a single bond or a C₁₋₂ linkage, which optionally contains a non-vicinal O, S, or N(R²³), R⁷ and R¹⁴ together form a single bond or a C₁ linkage, or R⁷ and R¹⁶ together form a single bond or a C₁ linkage, where R²³ is H, C₁₋₆ alkyl, C₁₋₄ alkaryl, C₁₋₄ alkheteroaryl, COR^{24b}, CO₂R^{24a}, CONR^{24a}R^{24b}, CSR^{24b}, COSR^{24a}, CSOR^{24a}, CSNR^{24a}R^{24b}, SO₂R^{24a}, or SO₂NR^{24a}R^{24b}, wherein R^{24a} is C₁₋₆ alkyl, C₆₋₁₂ aryl, C₁₋₄ alkaryl, C₁₋₉ heteroaryl, or C₁₋₄ alkheteroaryl, R^{24b} is H, C₁₋₆ alkyl, C₆₋₁₂ aryl, C₁₋₄ alkaryl, C₁₋₉ heteroaryl, or C₁₋₄ alkheteroaryl, or R^{24a} and R^{24b} together form a C₂₋₆ linkage, optionally containing a non-vicinal O; R⁸ is H, C₁₋₆ alkyl, C₁₋₄ alkaryl, C₁₋₄ alkheteroaryl, R⁸ and R⁹ together are =O or =N—OR¹⁸, where R¹⁸ is H, C₁₋₆ alkyl, C₁₋₄ alkaryl, or C₁₋₄ alkheteroaryl, or R⁸ and R¹² together form a single bond; R⁹ is H, C₁₋₆ alkyl, C₁₋₄ alkaryl, C₁₋₄ alkheteroaryl, or R⁹ and R⁸ together are =O or =N—OR¹⁸, where R¹⁸ is as previously defined; R¹⁰ is H, C₁₋₆ alkyl, C₁₋₄ alkaryl, C₁₋₄ alkheteroaryl, R¹⁰ and R⁷ together form a ring as previously defined, R¹⁰ and R¹¹ together are =O, R¹⁰ and R¹⁶ together form a C₁₋₂ alkyl linkage, which optionally contains a non-vicinal O, S, or N(R²³), or R¹⁰ and R¹⁷ together form a C₁₋₃ alkyl linkage, which optionally contains a non-vicinal O, S, or N(R²³), where R²³ is as previously defined; R¹¹ is H; R¹² is H, C₁₋₆ alkyl, C₁₋₄ alkaryl, C₁₋₄ alkheteroaryl, R¹² and R¹⁶ together form a C₂₋₄ alkyl linkage, which optionally contains a non-vicinal O, S, or N(R²³), or R¹² and R⁷ or R¹² and R⁸ together form a ring as previously defined; R¹³ is H, C₁₋₆ alkyl, C₁₋₄ alkaryl, or C₁₋₄ alkheteroaryl; R¹⁴ is H, C₁₋₆ alkyl, C₁₋₄ alkaryl, C₁₋₄ alkheteroaryl, or R¹⁴ and R⁷ together form a ring as previously defined; R¹⁵ is H, C₁₋₆ alkyl, C₁₋₄ alkaryl, or C₁₋₄ alkheteroaryl; R¹⁶ is H, C₁₋₆ alkyl, C₁₋₆ alkoxy, C₆₋₁₂ aryl, C₁₋₉ heteroaryl, C₁₋₄ alkaryl, C₁₋₄ alkheteroaryl, or R¹⁶ and R⁷, R¹⁶ and R¹⁰, or R¹⁶ and R¹² together form rings as previously defined; and R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl, or R¹⁷ and R¹⁰ together form a ring as previously defined.

In one embodiment, W is O; Y is H; A is OH, X is H or COCH₃, and each of R⁴ and R^{4′}, independently, is H or is: 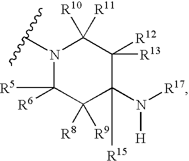
where each of R⁵ and R⁶ is H, or R⁵ and R⁶ together are =O, each of R⁸, R⁹, R¹², R¹³ and R¹⁵ is H, C₁₋₆ alkyl, or C₁₋₄ alkaryl, each of R¹⁰ and R¹¹ is H, C₁₋₆ alkyl, or C₁₋₄ alkaryl, or R¹⁰ and R¹¹ together are =O, R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl, and where R⁴ and R^{4′} cannot both be H at the same time.

In another embodiment, W is O; Y is H; A is H or OH, X is H or COCH₃, and each of R⁴ and R^{4′}, independently, is H or is: 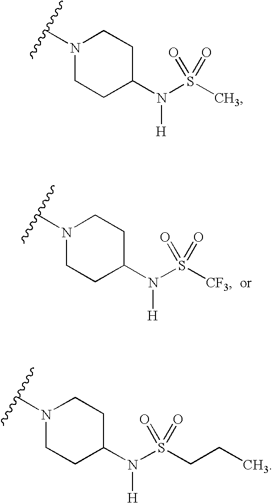
and where R⁴ and R^{4′} cannot both be H at the same time.

In another embodiment, W is O; Y is H; A is H or OH, X is H or COCH₃, and each of R⁴ and R^{4′}, independently, is H or is: 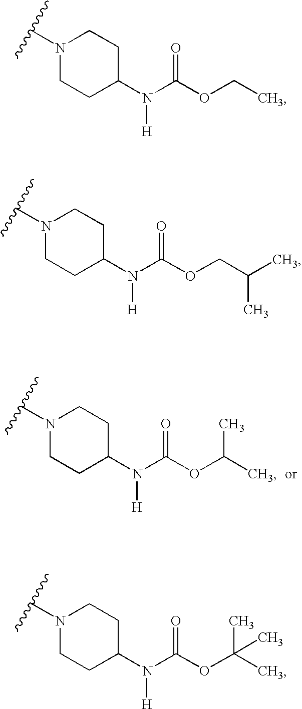
and where R⁴ and R^{4′} cannot both be H at the same time.

In yet another embodiment, W is O; Y is H; X is H or COCH₃, A is H or OH; and each of R⁴ and R^{4′}, independently, is H or is: 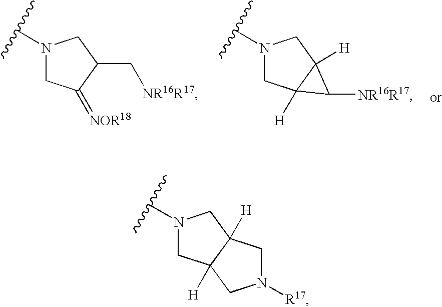
where R¹⁶ is H, C₁₋₆ alkyl, C₁₋₆ alkoxy, C₆₋₁₂ aryl, C₁₋₉ heteroaryl, C₁₋₄ alkaryl, or C₁₋₄ alkheteroaryl; R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl; and R¹⁸ is H, C₁₋₆ alkyl, C₁₋₄ alkaryl, or C₁₋₄ alkheteroaryl, and where R⁴ and R^{4′} cannot both be H at the same time.

Alternatively, for a compound of formula (IV), when m is 0 and n is 1 in the formula that represents R⁴ and/or R^{4′}: R⁷ and R¹⁰ together form a single bond or a C₁₋₄ linkage, which optionally contains a non-vicinal O, S, or N(R²³), R⁷ and R¹² together form a single bond or a C₁₋₃ linkage, which optionally contains a non-vicinal O, S, or N(R²³), or R⁷ and R¹⁴ together form a single bond or a C₁₋₂ linkage, which optionally contains a non-vicinal O, S, or N(R²³), where R²³ is as previously defined; each of R⁸ and R⁹ is H; R¹⁰ is H or R¹⁰ and R⁷ together form a single bond or a C₁₋₄ linkage, which optionally contains a non-vicinal O, S, or N(R²³), where R²³ is as previously defined; R¹¹ is H; R¹² is H, C₁₋₆ alkyl, C₁₋₄ alkaryl, C₁₋₄ alkheteroaryl, R¹² and R⁷ together form a single bond or a C₁₋₃ linkage, which optionally contains a non-vicinal O, S, or N(R²³), R¹² and R¹³ together form a —CH₂CH₂— linkage, or R¹² and R¹⁶ together form a C₂₋₄ alkyl linkage, which optionally contains a non-vicinal O, S, or N(R²³) , where R²³ is as previously defined; R¹³ is H, C₁₋₆ alkyl, C₁₋₄ alkaryl, C₁₋₄ alkheteroaryl, or R¹³ and R¹² together form a —CH₂CH₂— linkage; R¹⁴ is H, C₁₋₆ alkyl, C₁₋₄ alkaryl, C₁₋₄ alkheteroaryl, or R¹⁴ and R⁷together form a single bond or a C₁₋₂ linkage, which optionally contains a non-vicinal O, S, or N(R²³), where R²³ is as previously defined; R¹⁵ is H, C₁₋₆ alkyl, C₁₋₄ alkaryl, or C₁₋₄ alkheteroaryl; R¹⁶ is H, C₁₋₆ alkyl, C₁₋₆ alkoxy, C₆₋₁₂ aryl, C₁₋₉ heteroaryl, C₁₋₄ alkaryl, C₁₋₄ alkheteroaryl, or R¹⁶ and R¹² together form a C₂₋₄ alkyl linkage, which optionally contains a non-vicinal O, S, or N(R²³), where R²³ is as previously defined; and R¹⁷ is H, C₁₋₆ alkyl, C₁₋₄ alkaryl, C₁₋₄ alkheteroaryl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is as previously defined and where each alkyl linkage of 2 carbons or more may contain a non-vicinal O, S, or N(R²³) where R²³ is as previously defined.

In one embodiment, W is O; Y is H; X is H or COCH₃; A is H or OH; and each of R⁴ and R^{4′}, independently, is H or is: 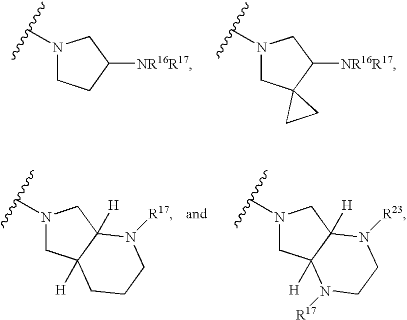
where R¹⁶ is H, C₁₋₆ alkyl, C₁₋₆ alkoxy, C₆₋₁₂ aryl, C₁₋₉ heteroaryl, C₁₋₄ alkaryl, or C₁₋₄ alkheteroaryl, and each of R¹⁷ and R²³ is as previously defined, and where R⁴ and R^{4′} cannot both be H at the same time.

Alternatively, for a compound of formula (IV), A is OH; X is H; W, and Y are as described above; and each of R⁴ and R^{4′}, independently, is H or is: 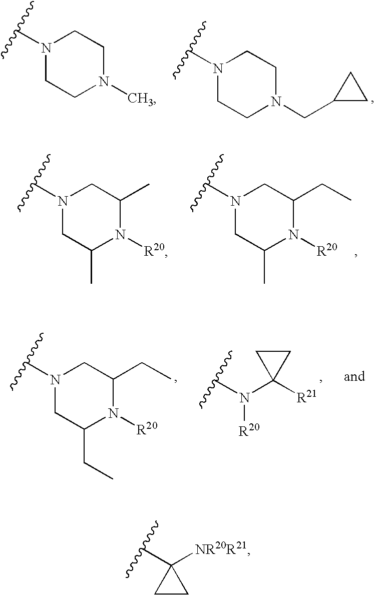
where R²¹ is H, C₁₋₆ alkyl, C₆₋₁₂ aryl, C₁₋₉ heteroaryl, C₁₋₄ alkaryl, or C₁₋₄ alkheteroaryl, R²⁰ is H, C₁₋₆ alkyl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl, and where R⁴ and R^{4′} cannot both be H at the same time.

Alternatively, A is OH; X is COCH₃; W, and Y are as defined above; and each of R⁴ and R^{4′}, independently, is H or is: 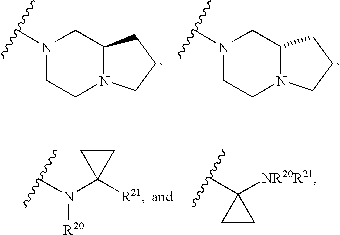
where R²¹ is H, C₁₋₆ alkyl, C₆₋₁₂ aryl, C₁₋₉ heteroaryl, C₁₋₄ alkaryl, or C₁₋₄ alkheteroaryl, R²⁰ is H, C₁₋₆ alkyl, COR¹⁹, CO₂R¹⁹, CONHR¹⁹, CSR¹⁹, COSR¹⁹, CSOR¹⁹, CSNHR¹⁹, SO₂R¹⁹, or SO₂NHR¹⁹, where R¹⁹ is C₁₋₆ alkyl, C₆₋₁₂ aryl, C₁₋₄ alkaryl, C₁₋₉ heteroaryl, or C₁₋₄ alkheteroaryl, and where R⁴ and R^{4′} cannot both be H at the same time.

Alternatively, A is H or OH; X is H or COCH₃; W, and Y are as defined above; and each of R⁴ and R^{4′}, independently, is H or is: 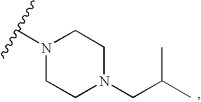
wherein R⁴ and R⁴′ cannot both be H at the same time.

Alternatively, A is H or OH; X is H or COCH₃; W and Y are as defined above; and each of R⁴ and R^{4′}, independently, is H or is: 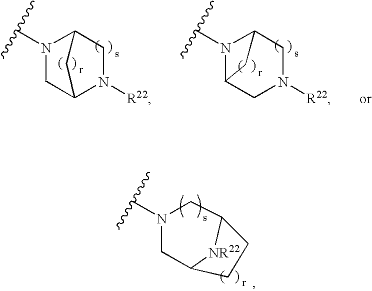
where R²² is H, C₁₋₆ alkyl, C₆₋₁₂ aryl, C₁₋₉ heteroaryl, C₁₋₄ alkaryl, C₁₋₄ alkheteroaryl, COR²⁴, CO₂R²⁴, CONHR²⁴, CSR²⁴, COSR²⁴, CSOR²⁴, CSNHR²⁴, SO₂R²⁴, or SO₂NHR²⁴, wherein R²⁴ is C₁₋₆ alkyl, C₆₋₁₂ aryl, C₁₋₄ alkaryl, C₁₋₉ heteroaryl, or C₁₋₄ alkheteroaryl, each of r and s is, independently, 1-2, and where R⁴ and R^{4′} cannot both be H at the same time.

Alternatively, A is H or OH; X is H or COCH₃; W and Y are as defined above; and each of R⁴ and R^{4′}, independently, is H or is: 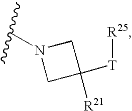
where T is O, S, NR²⁶, or a bond, where each of R²¹, R²⁵, and R²⁶ is H, C₁₋₆ alkyl, C₆₋₁₂ aryl, C₁₋₉ heteroaryl, C₂₋₉ heterocyclyl, C₁₋₄ alkaryl, or C₁₋₄ alkheteroaryl, or R²⁵ and R²⁶ together form a 3-8-membered ring, with the ring optionally containing a non-vicinal oxygen, and where R⁴ and R^{4′} cannot both be H at the same time.

Alternatively, A is H or OH; X is H or COCH₃; W and Y are as defined above; and each of R⁴ and R^{4′}, independently, is H or is: 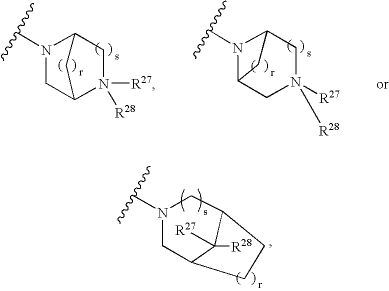
wherein R²⁷ is H, C₁₋₆ alkyl, C₁₋₄ alkaryl, or C₁₋₄ alkheteroaryl; R²⁸ is H, C₁₋₆ alkyl, C₆₋₁₂ aryl, C₁₋₉ heteroaryl, C₂₋₉ heterocyclyl, C₁₋₄ alkaryl, C₁₋₄ alkheteroaryl, OR^{24b}, or NR^{24a}R^{24b}, wherein R^{24a} is C₁₋₆ alkyl, C₆₋₁₂ aryl, C₁₋₄ alkaryl, C₁₋₉ heteroaryl, or C₁₋₄ alkheteroaryl, R^{24b} is H, C₁₋₆ alkyl, C₆₋₁₂ aryl, C₁₋₄ alkaryl, C₁₋₉ heteroaryl, or C₁₋₄ alkheteroaryl, or R^{24a} and R^{24b} together form a C₂₋₆ linkage, optionally containing a non-vicinal O; and each of r and s is, independently, 1-2, and where R⁴ and R^{4′} cannot both be H at the same time.

Alternatively, A is H or OH; X is H or COCH₃; W and Y are as defined above; and each of R⁴ and R^{4′}, independently, is H or is 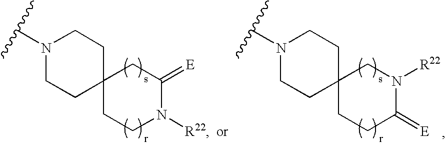
where =E is =O or (H,H), R²² is H, C₁₋₆ alkyl, C₆₋₁₂ aryl, C₁₋₉ heteroaryl, C₁₋₄ alkaryl, C₁₋₄ alkheteroaryl, COR²⁴, CO₂R²⁴, CONHR²⁴, CSR²⁴, COSR²⁴, CSOR²⁴, CSNHR²⁴, SO₂R²⁴, or SO₂NHR²⁴, where R²⁴ is C₁₋₆ alkyl, C₆₋₁₂ aryl, C₁₋₄ alkaryl, C₁₋₉ heteroaryl, or C₁₋₄ alkheteroaryl, r is 1-2, s is 0-1, and where R⁴ and R^{4′} cannot both be H at the same time.

Alternatively, A is H or OH; X is H or COCH₃; W and Y are as defined above; and each of R⁴ and R^{4′}, independently, is H or is: 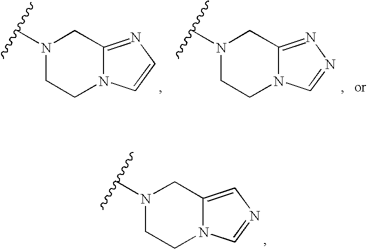
and where R⁴ and R⁴′ cannot both be H at the same time.

For those compounds in which R⁴ has the formula: 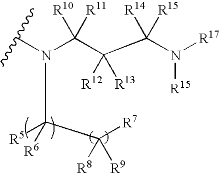
several different ring systems can be constructed from this generic formula. In one example, compounds having formula (A) are constructed when each of m and n is 1 and R⁷ forms a single bond with R¹⁴.

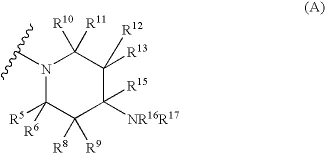

In another example, compounds having formula (B) are constructed when each of m and n is 1, R⁷ forms a single bond with R¹⁴, and R⁸ forms a single bond with R¹².

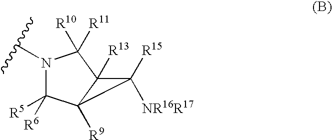

In another example, compounds having formula (C) are constructed when m is 0 and n is 1, R⁷ forms a single bond with R¹⁴, and R¹² forms a C₃ alkyl linkage with R¹⁶.

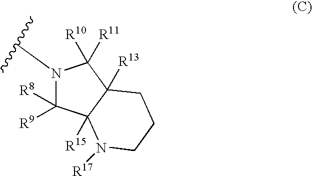

In another example, compounds having formula (D) are constructed when m is 0, n is 1, and R⁷ forms a single bond with R¹⁴.

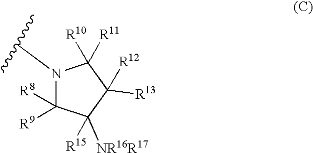

In another example, compounds having formula (E) are constructed when each of m and n is 1 and R⁷ forms a single bond with R¹².

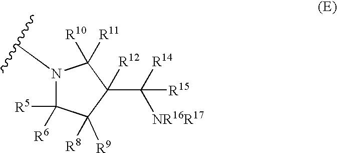

In another example, compounds having formula (F) are constructed when each of m and n is 1, R⁷ forms a single bond with R¹², and R⁸ forms a C₁ linkage with R¹⁶.

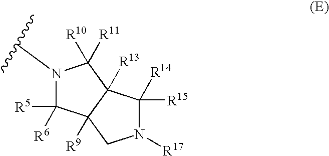

In yet another example, compounds having formula (G) are constructed when m is 0 and n is 1, R⁷ forms a single bond with R¹⁴, and R¹² forms a C₂ alkyl linkage, containing an NR²³ moiety, with R¹⁶.

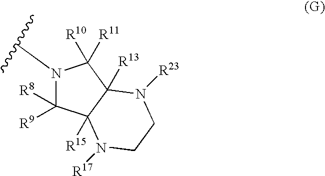

Rifamycins of Formula (V)

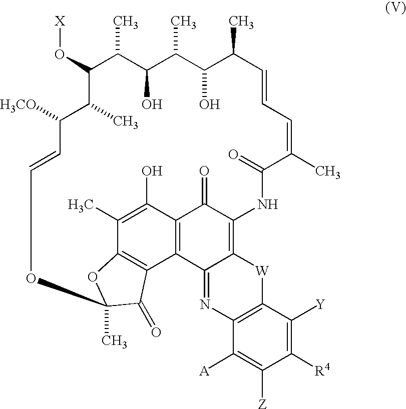

In formula (V), A is H, OH, O—(C₁₋₆ alkyl), O—(C₁₋₄ alkaryl), O—(C₆₋₁₂ aryl), O—-(C₁₋₉ heteroaryl), or O—(C₁₋₄ alkheteroaryl); W is O, S, or NR¹, wherein R¹ is H, C₁₋₆ alkyl, C₁₋₄ alkaryl, or C₁₋₄ alkheteroaryl; X is H or COR², wherein R² is C₁₋₆ alkyl, which can be substituted with 1-5 OH groups, O—(C₃₋₇ alkyl), which can be substituted with 1-4 OH groups, C₆₋₁₂ aryl, C₁₋₄ alkaryl, C₁₋₉ heteroaryl, or C₁₋₄ alkheteroaryl, wherein each alkyl carbon is bonded to no more than one oxygen atom; Y is H, Hal, or OR^{Y3}, wherein R^{Y3} is C₁₋₆ alkyl, C₆₋₁₂ aryl, C₁₋₄ alkaryl, C₁₋₉ heteroaryl, or C₁₋₄ alkheteroaryl; Z is H, Hal, or OR^{Z3}, wherein R^{Z3} is C₁₋₆ alkyl, C₆₋₁₂ aryl, C₁₋₄ alkaryl, C₁₋₉ heteroaryl, or C₁₋₄ alkheteroaryl; and

R⁴ has the formula:

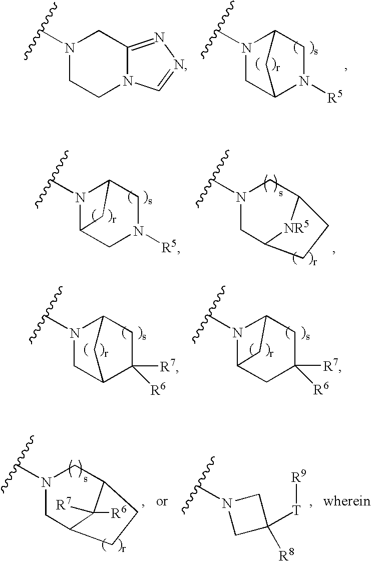

R⁵ is H, C₁₋₆ alkyl, C₁₋₄ alkaryl, C₁₋₄ alkheteroaryl, COR¹⁰, CO₂R¹¹, CONR¹⁰R¹¹ CSR¹⁰, COSR¹¹, CSOR¹¹, CSNR¹⁰R¹¹, SO₂R¹¹, or SO₂NR¹⁰OR¹¹, wherein R¹⁰ is H, C₁₋₆ alkyl, C₆-₁₂ aryl, C₁₋₄ alkaryl, C₁₋₉ heteroaryl, or C₁₋₄ alkheteroaryl, R¹¹ is C₁₋₆ alkyl, C₆₋₁₂ aryl, C₁₋₄ alkaryl, C₁₋₉ heteroaryl, or C₁₋₄ alkheteroaryl, or R¹⁰ and R¹¹ together form a C₂₋₆ linkage, optionally containing a non-vicinal O;

R⁶ is H, C₁₋₆ alkyl, C₁₋₄ alkaryl, or C₁₋₄ alkheteroaryl;

R⁷ is H, C₁₋₆ alkyl, C₆₋₁₂ aryl, C₁₋₉ heteroaryl, C₂₋₉ heterocyclyl, C₁₋₄ alkaryl, C₁₋₄ alkheteroaryl, OR¹², or NR¹²R¹³, where R¹² is H, C₁₋₆ alkyl, C₆₋₁₂ aryl, C₁₋₄ alkaryl, C₁₋₉ heteroaryl, or C₁₋₄ alkheteroaryl, R¹³ is C₁₋₆ alkyl, C₆₋₁₂ aryl, C₁₋₄ alkaryl, C₁₋₉ heteroaryl, or C₁₋₄ alkheteroaryl, or R¹² and R¹³ together form a C₂₋₆ linkage, optionally containing a non-vicinal O;

T is O, S, NR⁵, or a bond;

each of R⁸ and R⁹ is, independently, H, C₁₋₆ alkyl, C₆₋₁₂ aryl, C₁₋₉ heteroaryl, C₂₋₉ heterocyclyl, C₁₋₄ alkaryl, or C₁₋₄ alkheteroaryl, or R⁸ and R⁵ together form a 3-8-membered ring, with the ring optionally containing a non-vicinal oxygen;

and each of r and s is, independently, 1 or 2.

In one embodiment, the compound of formula (V) is one of the following compounds: 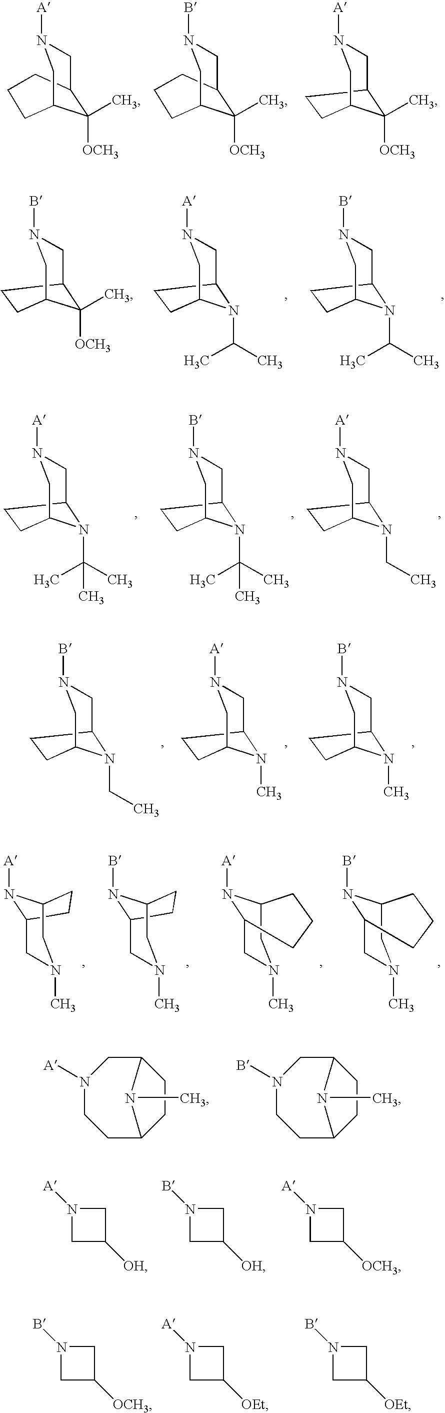 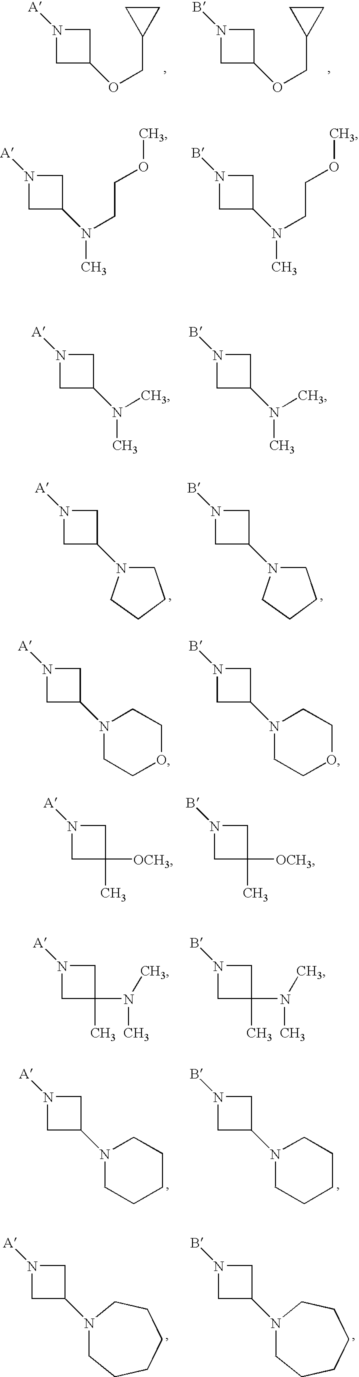 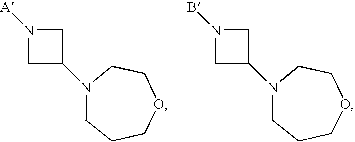
wherein A′ and B′ are as defined above.

Tables 1-4 give the structure and MIC values for some compounds of formulas (I)-(IV), respectively.

TABLE 1
Structures and MIC values of compounds of formula (I)

| | | | | MIC (μg/mL) | | | | |
|---|---|---|---|---|---|---|---|---|
| Compound | | | | S. | S. | E. | | |
| No. | Structure* | MW | MP (° C.) | aureus | pneumo. | faecalis | H. flu | E. coli |
| 1 | 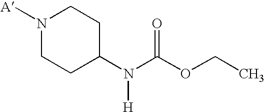 | 971.06 | 226-230 | 0.008 | 0.00025 | 2 | 2 | >8 |
| 2 | 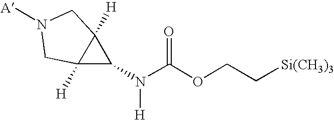 | 1041.23 | 230-231 | 1 | 0.03 | >8 | >8 | >8 |
| 3 | 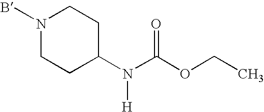 | 929.027 | 206-216 | 0.03 | 0.0005 | 0.12 | 0.25 | 4 |
| 4 | 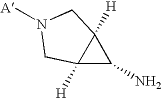 | 896.985 | >300 | 0.03 | 0.00025 | 0.5 | 0.25 | >8 |
| 5 | 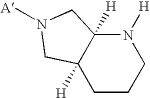 | 925.039 | >300 | 0.015 | 0.00025 | 0.5 | 2 | 8 |
| 6 | 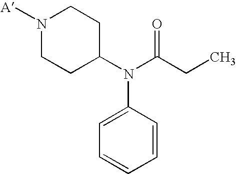 | 1031.16 | 224-228 | 0.015 | 0.00025 | 0.25 | 1 | 4 |
| 7 | 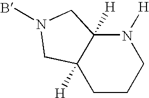 | 883.002 | 240-243 | 0.12 | 0.004 | 4 | 4 | >8 |
| 8 | 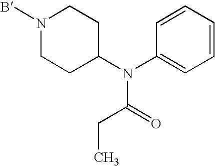 | 989.126 | 214-216 | 0.008 | 0.001 | 0.12 | 0.5 | 8 |
| 9 | 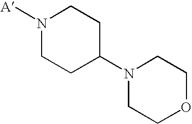 | 969.092 | 228-230 | 0.015 | 0.001 | 2 | 2 | 8 |
| 10 | 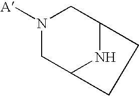 | 911.012 | 210-212 | 0.008 | 0.00012 | 1 | 2 | >8 |
| 11 | 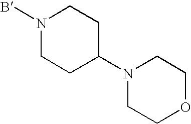 | 927.055 | 222-224 | 0.004 | 0.00012 | 0.25 | 1 | 4 |
| 12 | 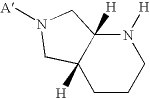 | 925.039 | >320 | 0.004 | 0.00025 | 0.12 | 0.25 | 8 |
| 13 | 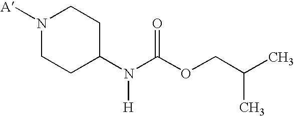 | 999.117 | 184-188 | 0.008 | 0.00012 | 0.5 | 2 | 4 |
| 14 | 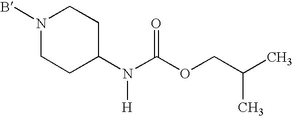 | 957.081 | 173-180 | | | | | |
| 15 | 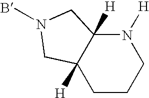 | 883.002 | 216-227 | 0.03 | 0.002 | 0.25 | 1 | >8 |
| 16 | 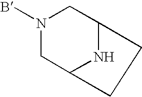 | 868.975 | 208-229 | 0.015 | 0.001 | 0.12 | 0.25 | 4 |
| 17 | 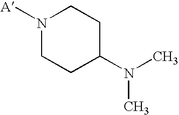 | 927.055 | >400 | 0.008 | 0.00012 | 1 | 1 | 8 |
| 18 | 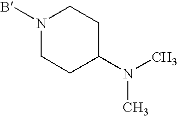 | 885.018 | 214-216 | 0.004 | 0.00012 | 0.12 | 0.5 | 4 |
| 19 | 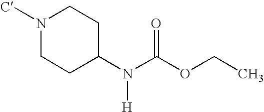 | 968.107 | 220-240 | 0.004 | 0.001 | 0.06 | 0.25 | 4 |
| 20 | 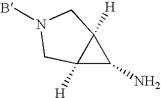 | 854.949 | 211-231 | 2 | 1 | >8 | >8 | >8 |
| 21 | 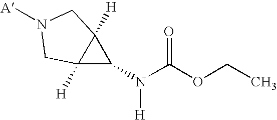 | 969.048 | >350 | 0.004 | 0.001 | 0.12 | 0.5 | 4 |
| 22 | 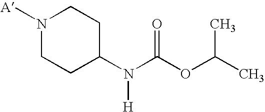 | 985.091 | >300 | 0.015 | 0.00025 | 4 | 2 | >8 |
| 23 | 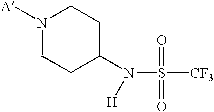 | 1031.06 | 238-240 | 0.015 | 0.00012 | 4 | 2 | >8 |
| 24 | 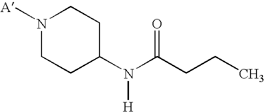 | 969.092 | 222-226 | 0.015 | 0.001 | 2 | 2 | >8 |
| 25 | 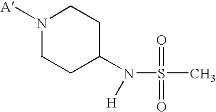 | 977.092 | 256-257 | 0.008 | 0.001 | 2 | 2 | >8 |
| 26 | 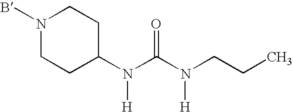 | 942.07 | 265-266 | 0.008 | 0.00012 | 2 | 1 | >8 |
| 27 | 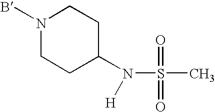 | 935.055 | 270-273 | 0.015 | 0.001 | 1 | 0.12 | >8 |
| 28 | 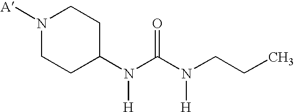 | 984.107 | 260-263 | 0.03 | 0.002 | 1 | 0.25 | >8 |
| 29 | 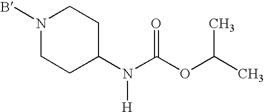 | 943.054 | 222-225 | 0.008 | 0.00012 | 2 | 1 | >8 |
| 30 | 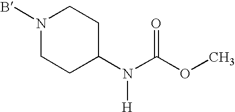 | 915 | 212-225 | 0.015 | 0.002 | 1 | 0.5 | >8 |
| 31 | 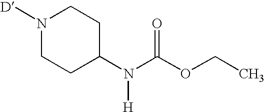 | 926.071 | 250-252 | 0.03 | 0.0005 | 0.25 | 0.25 | 8 |
| 32 | 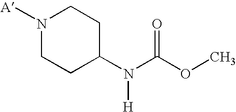 | 957.037 | 149-153 | 2 | 4 | >8 | >8 | >8 |
| 33 | 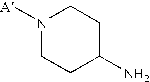 | 899.001 | 278-279 | 0.008 | 0.00006 | 1 | 1 | >8 |
| 34 | 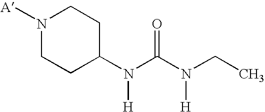 | 970.08 | 234-236 | 0.03 | 0.001 | 2 | 0.5 | 8 |
| 35 | 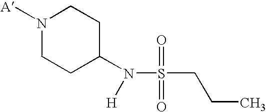 | 1005.15 | 220-226 | 0.015 | 0.00025 | 4 | 0.5 | 8 |
| 36 | 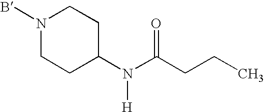 | 927.055 | 228-230 | 0.004 | 0.00003 | 2 | 0.25 | >8 |
| 37 | 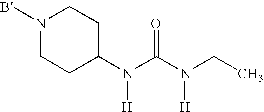 | 928.043 | 321-322 | 0.03 | 0.004 | 2 | 0.25 | >8 |
| 38 | 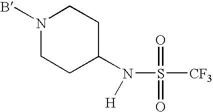 | 989.026 | 245-246 | 0.12 | 0.015 | 8 | 0.25 | >8 |
| 39 | 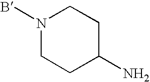 | 856.964 | 265-266 | 0.03 | 0.001 | 2 | 0.25 | >8 |
| 40 | 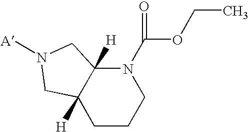 | 997.102 | >300 | 0.06 | 0.008 | 1 | 0.5 | >8 |
| 41 | 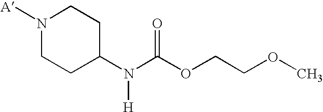 | 1001.09 | 175-183 | 0.25 | 0.0005 | >8 | 2 | >8 |
| 42 | 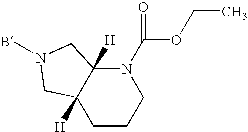 | 955.065 | >300 | 0.008 | 0.00012 | 2 | 0.5 | 8 |
| 43 | 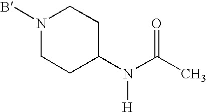 | 899.001 | 232-236 | 0.03 | 0.002 | 2 | 0.5 | >8 |
| 44 | 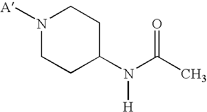 | 941.038 | 232-234 | 0.06 | 0.008 | 2 | 0.25 | >8 |
| 45 | 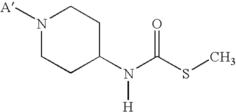 | 973.104 | 150-155 | 0.015 | 0.001 | 4 | 0.5 | 8 |
| 46 | 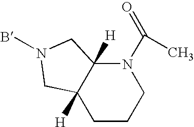 | 925.039 | 148-150 | 0.004 | 0.00006 | 2 | 0.5 | >8 |
| 47 | 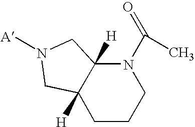 | 967.076 | 148-149 | 0.008 | 0.002 | 1 | 0.25 | >8 |
| 48 | 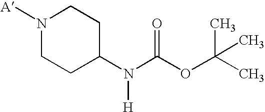 | 999.117 | 218-228 | 0.008 | 0.00025 | 2 | 1 | >8 |
| 49 | 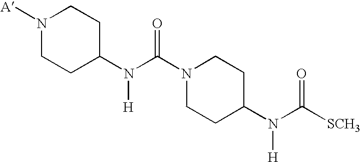 | 1099.2 | >300 | 0.06 | 0.0005 | 8 | 2 | >8 |
| 50 | 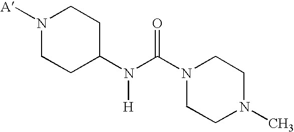 | 1025.16 | >300 | 0.03 | 0.00012 | 4 | 0.5 | >8 |
| 51 | 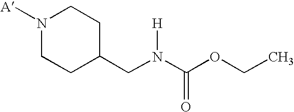 | 985.091 | 175-185 | 0.06 | 0.002 | 8 | 0.25 | 8 |
| 52 | 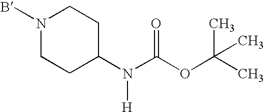 | 957.081 | 230-232 | 0.015 | 0.00025 | 4 | 0.12 | >8 |
| 53 | 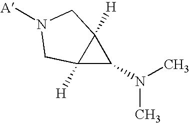 | 925.039 | >370 | 0.06 | 0.008 | 4 | 2 | >8 |
| 54 | 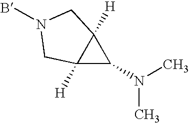 | 883.002 | >330 | 0.008 | 0.0005 | 2 | 0.25 | >8 |
| 55 | 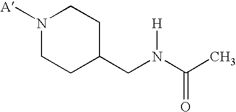 | 955.065 | 214-218 | 0.008 | 0.00025 | 1 | 0.5 | 8 |
| 56 | 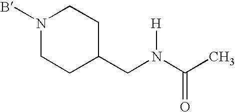 | 913.028 | 205-213 | 0.004 | 0.001 | 0.12 | 0.25 | 8 |
| 57 | 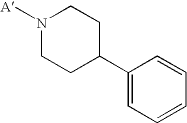 | 960.084 | 220-222 | 0.008 | 0.0005 | 2 | 0.5 | 8 |
| 58 | 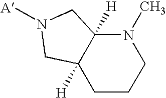 | 939.066 | >300 | 0.03 | 0.004 | 1 | 0.25 | >8 |
| 59 | 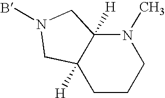 | 897.029 | >300 | 0.004 | 0.001 | 0.06 | 0.12 | 4 |
| 60 | 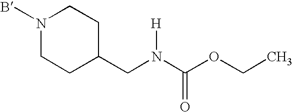 | 943.054 | 110-195 | 0.008 | 0.0005 | 0.5 | 0.06 | >8 |
| 61 | 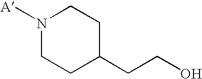 | 928.039 | 205-212 | 0.004 | 0.00012 | 1 | 0.25 | >8 |
| 62 | 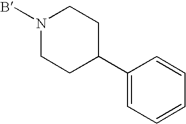 | 918.047 | 228-230 | 0.015 | 0.008 | 8 | 0.5 | >8 |
| 63 | 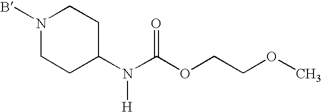 | 959.053 | 210-214 | 0.008 | 0.002 | 1 | 0.25 | 8 |
| 64 | 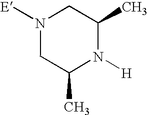 | 913.096 | >250 | 0.004 | 0.00012 | 0.12 | 0.25 | 4 |
| 65 | 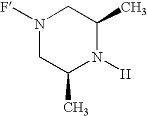 | 871.059 | >300 | 0.002 | 0.00025 | 0.12 | 0.12 | 2 |
| 66 | 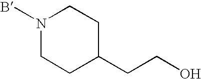 | 886.002 | 115-122 | 0.004 | 0.0005 | 0.12 | 0.12 | 4 |
| 67 | 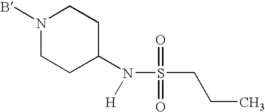 | 963.109 | | 0.008 | 0.001 | 0.5 | 0.12 | >8 |
| 68 | 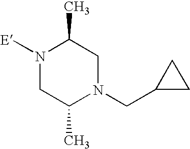 | 967.187 | >250 | 0.06 | 0.0005 | 2 | 0.5 | >8 |
| 69 | 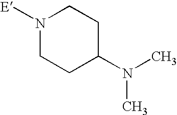 | 927.123 | >250 | 0.015 | 0.00025 | 0.5 | 0.5 | 4 |
| 70 | 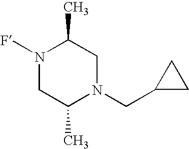 | 925.151 | >250 | 0.03 | 0.001 | 1 | 1 | >8 |
| 71 | 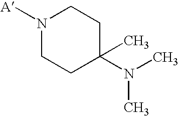 | 941.082 | 216-220 | 0.008 | 0.00012 | 0.25 | 0.5 | 4 |
| 72 | 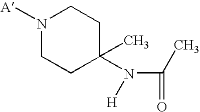 | 955.065 | 210-214 | 0.008 | 0.00025 | 2 | 0.5 | >8 |
| 73 | 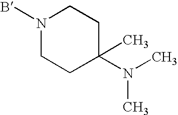 | 899.045 | 215-218 | 0.002 | 0.00025 | 0.03 | 0.12 | 2 |
| 74 | 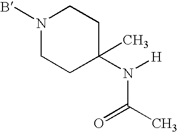 | 913.028 | 220-221 | 0.03 | 0.001 | 0.5 | 0.12 | >8 |
| 75 | 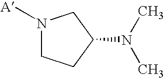 | 913.028 | | 0.002 | 0.00012 | 0.12 | 0.25 | 4 |
| 76 | 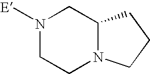 | 925.107 | >250 | 0.008 | 0.00025 | 0.5 | 0.25 | 4 |
| 77 | 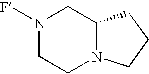 | 883.07 | >250 | 0.002 | 0.00025 | 0.25 | 0.12 | 2 |
| 78 | 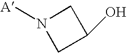 | 875.99 | | 0.002 | 0.00012 | 0.12 | 0.12 | 4 |
| 79 | 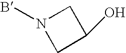 | 833.95 | | | | | | |

*A′, B′, C′, D′, B′, and F′ are defined above.

TABLE 2
Structures and MIC values of compounds of formula (II)

MIC (μg/mL)

| Cmpd. | | | | | C. tracho- | S. | S. | F. | H. | E. |
|---|---|---|---|---|---|---|---|---|---|---|
| No. | Structure* | Formula | MP (° C.) | MW | matis | aureus | pneumo. | faecalis | flu | coli |
| 80 | 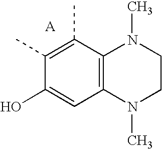 | C₄₇H₅₆N₄O₁₃ | 276—7 | 884.974 | 6.4 × 10⁻⁵ | 0.008 | 0.00012 | 1 | 1 | 8 |
| 81 | 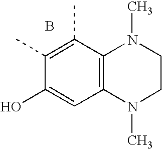 | C₄₅H₅₄N₄O₁₂ | 234—40 | 842.938 | 0.001 | 0.5 | 0.06 | 1 | 2 | 8 |
| 82 | 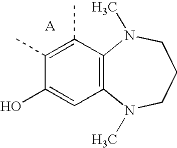 | C₄₈H₅₈N₄O₁₃ | 236—40 | 899.001 | 6.4 × 10⁻⁵ | 0.06 | 0.008 | 4 | 2 | >8 |
| 83 | 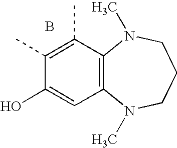 | C₄₆H₅₆N₄O₁₂ | 225—30 | 856.964 | 0.001 | 0.12 | 0.00012 | 1 | 2 | >8 |
| 84 | 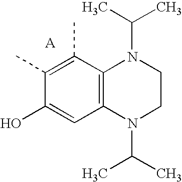 | C₅₁H₆₄N₄O₁₃ | | 941.082 | 0.01 | 0.06 | 0.008 | 4 | 2 | >8 |
| 85 | 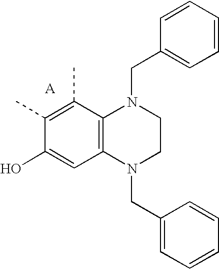 | C₅₉H₆₄N₄O₁₃ | | 1037.17 | | 2 | 0.12 | 8 | >8 | >8 |

*A and B represent the following moieties:
A is

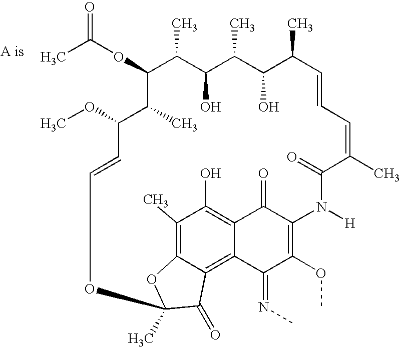

and B is

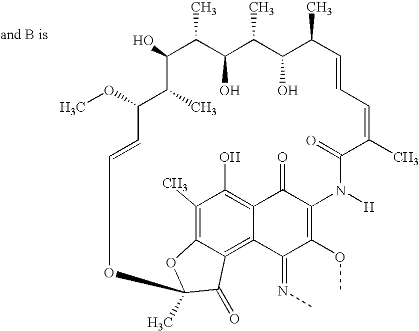

TABLE 3
Structures and MIC values of compounds of formula (III)

| | | MIC (μg/mL | | | | |
|---|---|---|---|---|---|---|
| Compound | | S. | S. | E. | | |
| No. | Structure* | aureus | pneumo. | faecalis | H. flu | E. coli |
| 86 | 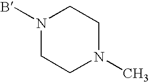 | 0.002 | 0.00012 | 0.015 | 0.25 | 4 |
| 87 | 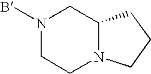 | 0.002 | 0.00025 | 0.03 | 0.25 | 4 |
| 88 | 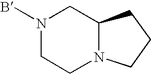 | 0.004 | 0.00025 | 0.03 | 0.25 | 8 |
| 89 | 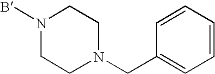 | 0.03 | 0.001 | >8 | 8 | >8 |
| 90 | 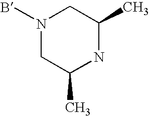 | 0.008 | 0.0005 | 0.06 | 0.25 | 4 |
| 91 | 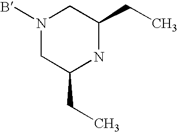 | 0.03 | 0.0005 | 0.12 | 0.25 | 4 |
| 92 | 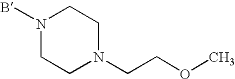 | 0.004 | 0.00025 | 0.12 | 0.25 | 8 |
| 93 | 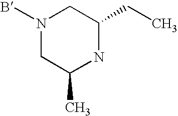 | 0.004 | 0.00012 | 0.5 | 0.25 | 4 |
| 94 | 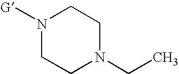 | 0.008 | 0.0005 | 0.5 | 0.25 | 8 |
| 95 | 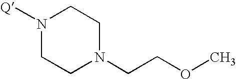 | 0.12 | 0.001 | 8 | 2 | >8 |
| 96 | 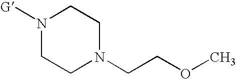 | 0.015 | 0.005 | 1 | 0.5 | >8 |
| 97 | 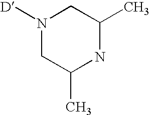 | 1 | 2 | >8 | 4 | >8 |
| 98 | 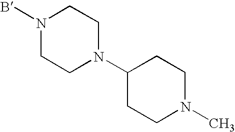 | 0.015 | 0.002 | 0.5 | 1 | >8 |
| 99 | 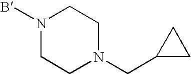 | 0.008 | 0.0005 | 0.06 | 0.25 | 2 |
| 100 | 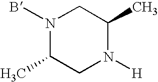 | 0.004 | 0.0005 | 0.12 | 0.25 | 4 |
| 101 | 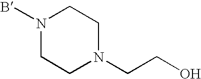 | 0.008 | 0.0005 | 0.12 | 0.25 | 8 |
| 102 | 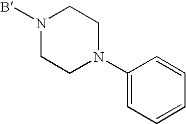 | 0.004 | 0.00 1 | 0.06 | 0.25 | 4 |
| 103 | 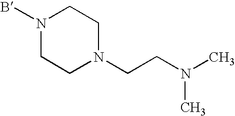 | 0.03 | 0.008 | 0.5 | 0.12 | >8 |
| 104 | 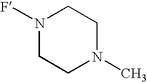 | 0.002 | 0.00012 | 0.12 | 0.12 | 2 |
| 105 | 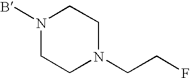 | 0.004 | 0.001 | 0.12 | 0.12 | 8 |
| 106 | 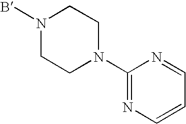 | 0.004 | 0.0005 | 0.25 | 0.12 | >8 |
| 107 | 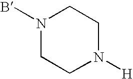 | 0.008 | 0.002 | 0.06 | 0.12 | 4 |
| 108 | 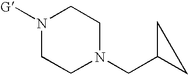 | 0.03 | 0.015 | 0.5 | 1 | >8 |
| 109 | 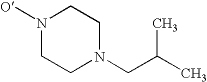 | 0.03 | 0.008 | 0.5 | 0.5 | 8 |
| 110 | 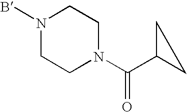 | 0.015 | 0.004 | 0.5 | 0.25 | 8 |
| 111 | 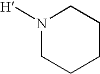 | 1 | 0.12 | 4 | 2 | 1 |
| 112 | 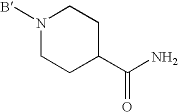 | 0.03 | 0.002 | 1 | 0.25 | >8 |
| 113 | 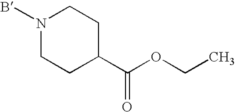 | 0.004 | 0.008 | 0.5 | 0.12 | >8 |
| 114 | 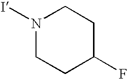 | 0.03 | 0.008 | 0.5 | 0.12 | 8 |
| 115 | 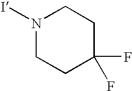 | 0.03 | 0.015 | 1 | 0.25 | >8 |
| 116 | 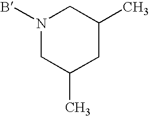 | 0.008 | 0.002 | 1 | 0.12 | >* |
| 117 | 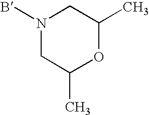 | 0.015 | 0.002 | 0.25 | 0.25 | 8 |
| 118 | 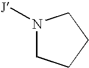 | 2 | 0.25 | >8 | 8 | >8 |
| 119 | 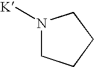 | 0.5 | 0.5 | 2 | 2 | >8 |
| 120 | 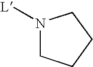 | 0.5 | 0.06 | 4 | 1 | >8 |
| 121 | 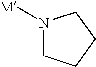 | 0.06 | 0.03 | 2 | 0.12 | 8 |
| 122 | 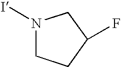 | 0.03 | 0.015 | 0.5 | 0.06 | 4 |
| 123 | 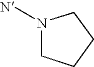 | 0.03 | 0.06 | 1 | 0.12 | 8 |
| 124 | 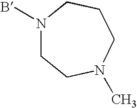 | 0.004 | 0.00012 | 0.03 | 0.25 | 4 |
| 125 | 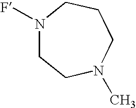 | 0.002 | 0.00025 | 0.25 | 0.12 | 2 |
| 126 | 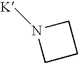 | 0.03 | 0.004 | 1 | 0.25 | 8 |
| 127 | 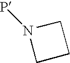 | 0.5 | 0.06 | 4 | 1 | >8 |
| 128 | 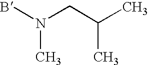 | 0.015 | 0.002 | 0.5 | 0.5 | >8 |
| 129 | 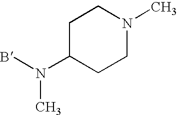 | 0.004 | 0.00025 | 0.5 | 0.25 | 4 |
| 130 | 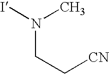 | 0.06 | 0.008 | 0.5 | 0.12 | 8 |
| 131 | 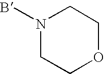 | 0.004 | 0.00025 | 0.06 | 0.12 | >8 |
| 132 | 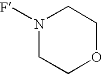 | 0.015 | 0.001 | 0.5 | 0.06 | 4 |
| 133 | 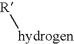 | 0.12 | 0.25 | 1 | 0.25 | >8 |
| 134 | 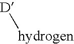 | 0.5 | 0.5 | 2 | 2 | 8 |
| 135 | 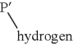 | 0.06 | 0.03 | 2 | 0.5 | >8 |
| 136 | 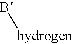 | 0.004 | 0.002 | 0.12 | 0.25 | 8 |
| 137 | 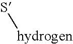 | 0.12 | 0.12 | 1 | 2 | >8 |
| 138 | 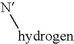 | 0.03 | 0.06 | 0.5 | 0.5 | >8 |
| 139 | 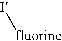 | 0.03 | 0.03 | 0.5 | 0.5 | >8 |

*A′, B′, C′, D′, E′, F′, G′, H′, I′, J′, K′, L′, M′, N′, O′, P′, Q′, R′, and S′ are defined above.

TABLE 4
Structures and MIC values of compounds of formula (IV)

| | | | | MIC (μg/mL) | | | | |
|---|---|---|---|---|---|---|---|---|
| Compound | | | | S. | S. | E. | | |
| No. | Structure* | MW | MP (° C.) | aureus | pneumo. | faecalis | H. flu | E. coli |
| 140 | 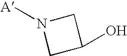 | 875.99 | | 0.002 | 0.00012 | 0.12 | 0.12 | 4 |
| 141 | 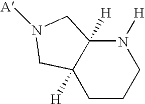 | 925.039 | >300 | 0.015 | 0.00025 | 0.5 | 2 | 8 |
| 142 | 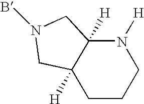 | 883.002 | 240-243 | 0.12 | 0.004 | 4 | 4 | >8 |
| 143 | 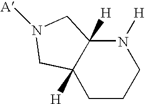 | 925.039 | >320 | 0.004 | 0.00025 | 0.12 | 0.25 | 8 |
| 144 | 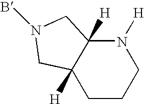 | 883.002 | 216-227 | 0.03 | 0.002 | 0.25 | 1 | >8 |
| 145 | 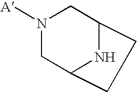 | 911.012 | 210-212 | 0.008 | 0.00012 | 1 | 2 | >8 |
| 146 | 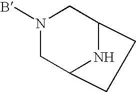 | 868.975 | 208-229 | 0.015 | 0.001 | 0.12 | 0.25 | 4 |
| 147 | 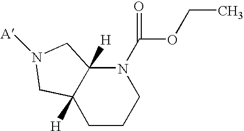 | 997.102 | >300 | 0.06 | 0.008 | 1 | 0.5 | >8 |
| 148 | 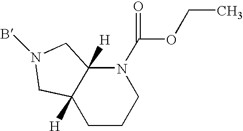 | 955.065 | >300 | 0.008 | 0.00012 | 2 | 0.5 | 8 |
| 149 | 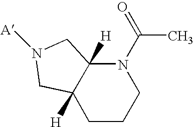 | 967.076 | 148-149 | 0.008 | 0.002 | 1 | 0.25 | >8 |
| 150 | 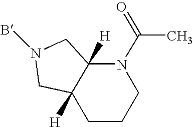 | 925.039 | 148-150 | 0.004 | 0.00006 | 2 | 0.5 | >8 |

*A′ and B′ are defined above.

### EXAMPLES

The following examples are intended to illustrate the invention. They are not meant to limit the invention in any way.

### Example 1

### Antimicrobial Susceptibility

To be successful in treating device-associated infection, an antimicrobial agent must possess antibacterial activity against surface-adhering microorganisms in the stationary growth phase. Therefore, the in vitro susceptibility of stationary growth phase S.aureus to the antimicrobials levofloxacin (a quinolone), rifampin, and the rifamycin Compounds 86, 151, and 152 is compared in Table 5.

The structure of Compound 86 is provided above. Compounds 151 and 152 have the following structures:

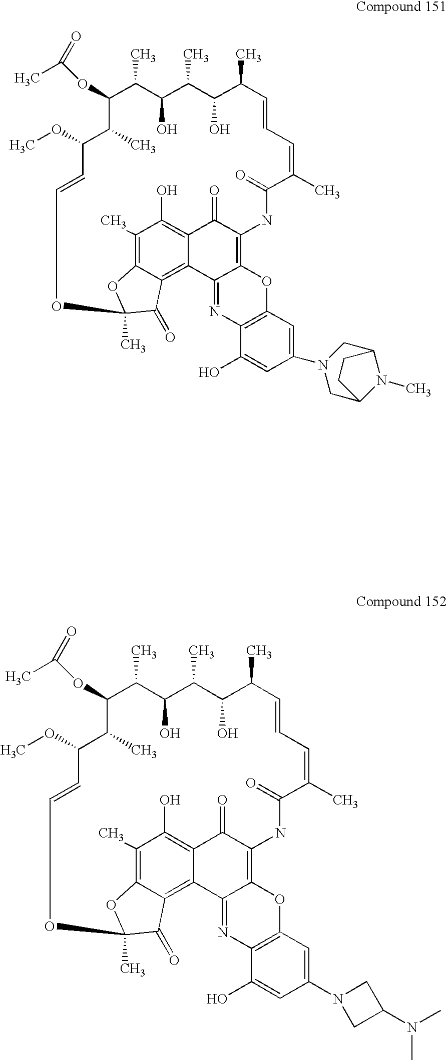

The minimal bactericidal concentration in the stationary growth phase (MBCₛₜₐₜ) was determined by using overnight bacterial cultures which were centrifuged and resuspended in medium containing 1% glucose supplemented phosphate buffered saline (PBS) pH 7.4 with 4% Muller Hinton Broth (Zimmerli et al., J Antimicrob. Chemother. 33:959-967 (1994)). In this medium, bacterial counts remained stable in the absence of antibacterial agents for >36 hours.

Compounds 86, 151, and 152, had the lowest MBCₛₜₐₜ values against tested S. aureus. These compounds were approximately 8-23 times more effective then rifampin, and 85-256 times more effective then levofloxacin, against stationary growth phase S. aureus in vitro (Table 5).

TABLE 5
In vitro susceptibility of S. aureus.

| Drug | MBCₛₜₐₜ^{a} (μg/ml) | Peak^{b}/MBCₛₜₐₜ ratio |
|---|---|---|
| Levofloxacin | 40 | 0.02 |
| Rifampin | 3.6 | 0.27 |
| Compound 86 | 0.313 | 3.61 |
| Compound 151 | 0.156 | |
| Compound 152 | 0.469 | |

^{a}MBCₛₜₐₜ, minimal bactericidal concentration in the stationary growth phase.
^{b}Peak concentration after a single intraperitoneal dose of 5 mg/kg (for levofloxacin) or 12.5 mg/kg (for rifampin and Compound 86).

### Example 2

### Pharmacokinetic Studies

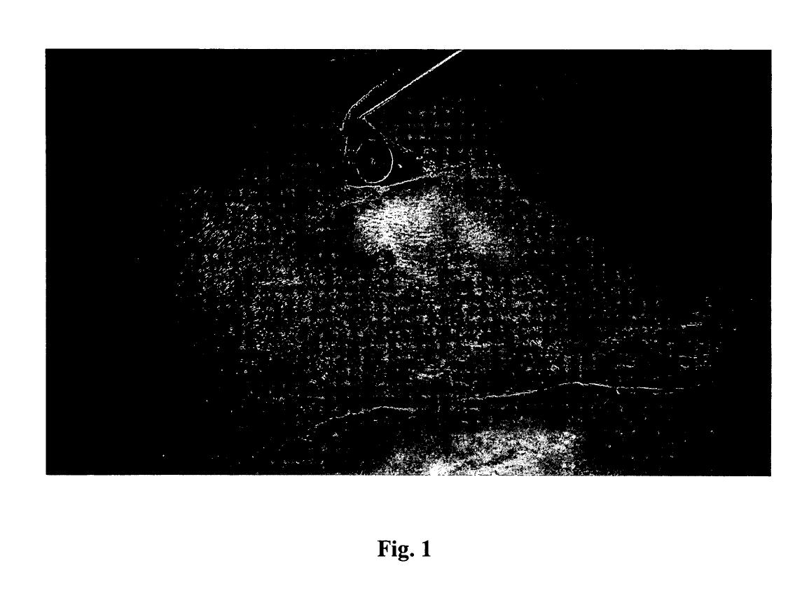

The pharmacokinetic profile of the various antimicrobial compounds was studied in a foreign-body infection model in guinea pigs (FIG. 1), as previously described (Blaser et al., Antimicrob. Agents Chemother. 39:1134-1139 (1995)). Teflon tissue cages were implanted into the flanks of guinea pigs. For pharmacokinetic studies, non-infected animals were used. Samples of cage fluid were aspirated by percutaneous cage puncture from non-infected animals at various times for up 12 hours following intraperitoneal administration of a single dose of Compound 86 or rifampin (12.5 mg/kg), or levofloxacin (5 mg/kg), or multiple dosing of rifampin and Compound 86 administered every 12 hours for four days (12.5 mg/kg). In addition, samples were taken once daily on subsequent days, just prior to dosing for rifampin and Compound 86 so that trough concentrations of antibacterials could be determined. Cage fluid concentration of Compound 86 and rifampin were determined by agar diffusion bioassays as described previously using Streptococcus pneumoniae ATCC 49619 or Escherichia coli V6311/65 as indicator organisms respectively (Klein et al., Antibiotics in laboratory medicine. p. 290-364 (2005)).

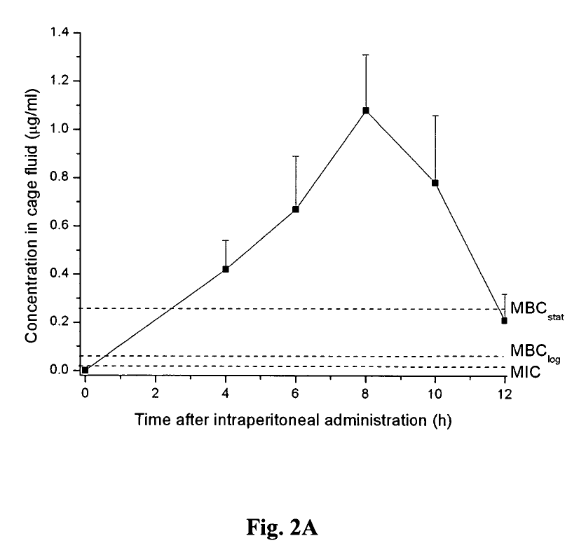

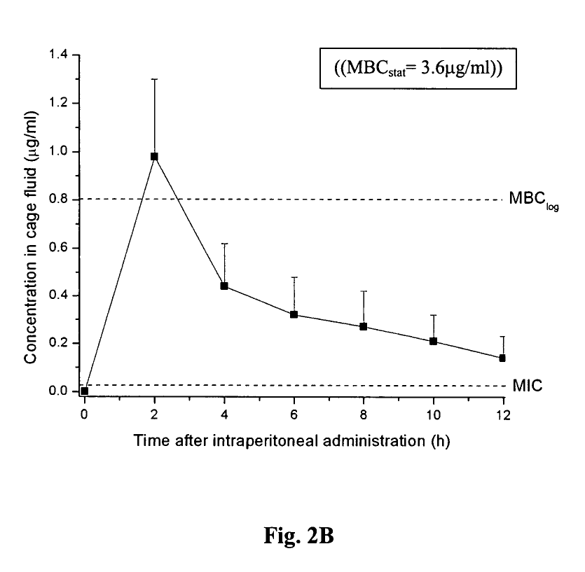

To understand the relative efficacy of peak drug concentration, it is compared to the minimal inhibitory concentration (MIC), the minimal bactericidal concentration for logarithmic phase growth (MBC_{log}) and the MBCₛₜₐₜ. The MIC was determined by broth dilution method with a standard inoculum of S. aureus ATCC29213 at 5×10⁵ CFU/ml. The MBC_{log} was defined as antimicrobial concentration that reduced the original inoculum by <99.9% after 24 hour incubation (i.e. 3 log 10 CFU/ml), as described in the Manual of Clinical Microbiology. The MBCₛₜₐₜ was determined as described in Example 1. The peak drug concentration of Compound 86 (1.13 μg/ml) in cage fluid from non-infected animals after single dose of 12.5 mg/kg was well above the minimal inhibitory concentration (MIC), the MBC_{log}, and the MBCₛₜₐₜ (Table 6 and FIG. 2B). This is in comparison to that of rifampin, in which the peak drug concentration (0.98 μg/ml) was above the MIC and the MBC_{log}, yet below that of the MBCₛₜₐₜ (FIG. 2A). The single dose pharmacokinetic data is also linked to the in vitro susceptibility data (Table 5) showing an increased Peak/MICᵣₐₜᵢₒ and Peak/MBCₛₜₐₜ ratio for Compound 86 in comparison to rifampin or levofloxacin. It is therefore anticipated that effective concentrations of Compound 86 are achieveable against adherent and stationary-phase infections. The exposure of Compound 86 following this single dose was similar to that of rifampin (AUC at 12 hours of 6.53 and 4.56 for Compound 86 and rifampin, respectively). The peak concentration of rifampin was achieved after a single intraperitoneal dose after two hours, whereas Compound 86 had a longer time to peak (Tₘₐₓ 8 h, Table 6). The half-life of rifampin was calculated to be 5.8 hours. Therefore, because of its longer Tₘₐₓ, the half-life of Compound 86 is anticipated as longer than that of rifampin, based on trough levels following multiple dosing.

TABLE 6
Pharmacokinetics in cage fluid after intraperitoneal administration of
antimicrobials in non-infected animals^{a}

| | | Dose | Cₘₐₓ | Cₘᵢₙ | Tₘₐₓ |
|---|---|---|---|---|---|
| Dosing | Antibiotic | (mg/kg) | (μg/ml) | (μg/ml)^{b} | (h) |
| Single | Levofloxacin | 5 | 0.97 ± 0.20 | 0.01 ± 0.01 | 2 |
| dose | Rifampin | 12.5 | 0.98 ± 0.32 | 0.14 ± 0.09 | 2 |
| | Compound 86 | 3 | 0.11 ± 0.03 | 0.04 ± 0.01 | 10 |
| | Compound 86 | 12.5 | 1.13 ± 0.23 | 0.14 ± 0.11 | 8 |
| Multiple | Rifampin | 12.5 | 0.61 ± 0.13 | 0.22 ± 0.09 | 4 |
| dose^{c} | Compound 86 | 12.5 | 0.82 ± 0.49 | 0.41 ± 0.09 | 6 |

^{a}Concentration values are means ± SD from 12 cage fluid aspirates.
^{b}Cₘᵢₙ (trough concentration) was measured 12 h after dosing.
^{c}The indicated dose was administered every 12 h for 4 days; the pharmacokinetic values were determined on day 4.

### Example 3

### Antimicrobial Treatment Studies

A foreign-body infection model in guinea pigs was used for in vivo analysis of antimicrobials as described in Example 2, however in this example the animals were infected. Cages were infected by percutaneous inoculation (200 μl) of a stationary overnight culture containing 2×10⁴ CFU S. aureus. Antimicrobial treatment was initiated 24 hours after cage infection (day 1). Animals were randomized into eight treatment groups: control (saline), levofloxacin 5 mg/kg, rifampin 12 mg/kg (with and without levofloxacin 5 mg/kg), Compound 86 at 3 mg/kg and 12 mg/kg (each dose with and without levofloxacin 5 mg/kg). Antibiotics were administered intraperitoneally every 12 hours for four days (total eight doses). Quantitative cultures of aspirated cage fluid were performed immediately before the initiation of antimicrobial treatment ((day 1)), during the treatment before the last antimicrobial dose (day 4) and 5 days after completion of treatment (day 9). On day 9, cages were removed, and presence of bacteria was evaluated to establish a cure rate.

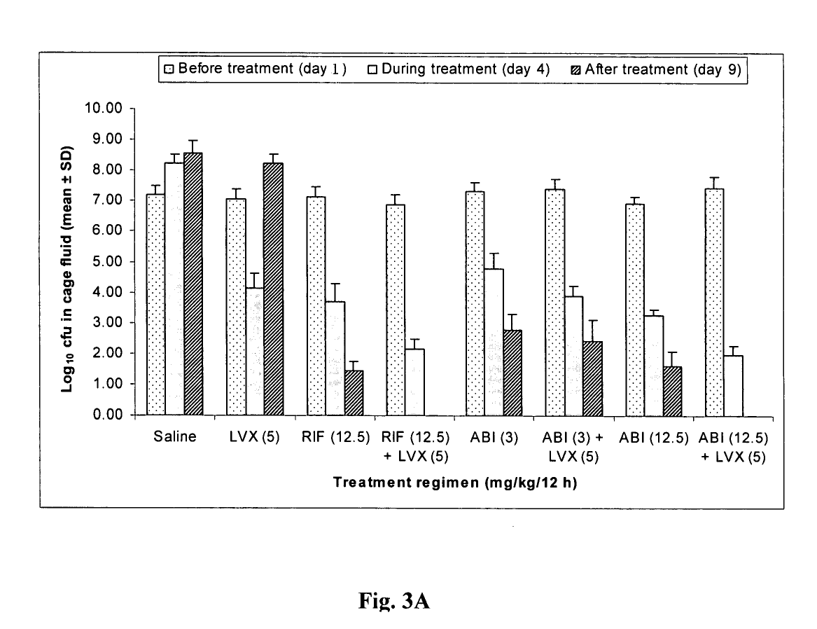

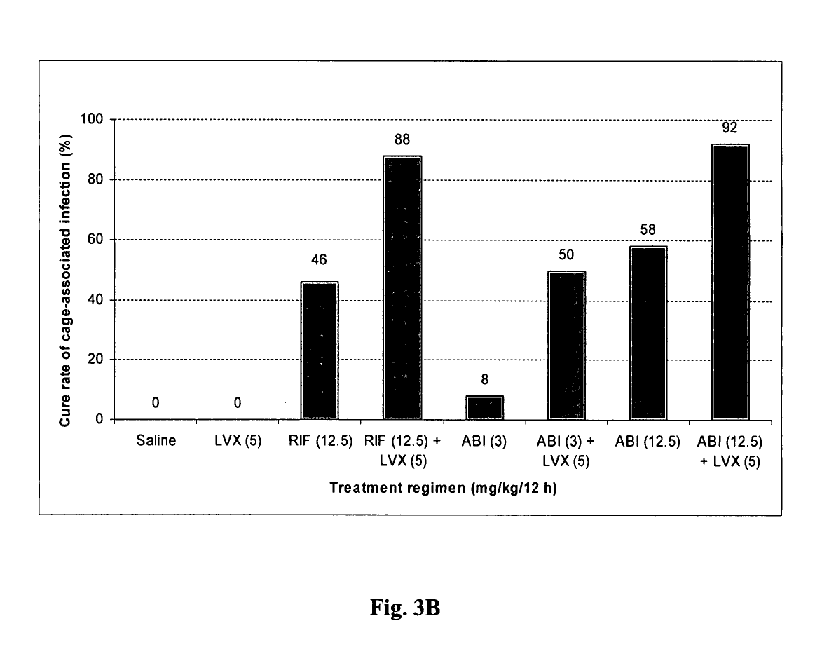

The titer of bacteria was undetectable prior to infection, and increased to ≈10⁷ CFU/ml of S. aureus 24 hours after inoculation in all cage fluid samples (FIG. 3A, dotted bars). In the cages of infected, untreated control animals, bacterial counts increased during the course of infection to >10⁸ CFU/ml cage fluid (FIG. 3A, Saline, grey bars). No spontaneous cure of cage-associated infection occurred in untreated cages through the entire nine days of the study (FIG. 3B, Saline). Levofloxacin alone reduced the bacterial count during treatment (FIG. 3A, LVX (5), grey bars), but bacteria regrew to similar counts as untreated controls after treatment (FIG. 3A, LVX (5), horizontal bars); no cage was cured (FIG. 3B, LVX (5)). Rifampin alone showed a cure rate of 46% (FIG. 3B, RIF (12.5)), which was further improved to 88% by the addition of levofloxacin (FIG. 3B, RIF (12.5)+LVX (5), p<0.05). Similarly, exposure to Compound 86 (ABI) alone resulted in a cure rate of 58% (FIG. 3B, ABI (12.5)), compared with 92% for Compound 86 and levofloxacin in combination (FIG. 3B, ABI (12.5)+LVX (5), p<0.16). The efficacy of high-dose therapy (12.5 mg/kg) of rifampin or Compound 86 was similar (46% and 58% respectively) and superior to the low-dose therapy (3 mg/kg) of Compound 86 (FIG. 3B, ABI (3)). Treatment outcome data is summarized in Table 7, clearly showing the efficacy of Compound 86, and Compound 86 combination treatment with a second antibiotic, in comparison to other antimicrobial treatment regimen.

TABLE 7
Treatment outcome of cage-associated infection with S. aureus

| | Mean log₁₀ CFU/ml ± SD | | Cure^{c} rate |
|---|---|---|---|
| Drug (dose in | in aspirated cage fluid^{b} | | of removed |
| mg/kg/12 h) | During treatment | After treatment | cages |
| Control (saline) | 8.23 ± 0.29 (0/12) | 8.57 ± 0.38 (0/12) | 0% (0/12) |
| Levofloxacin | 4.13 ± 0.50 (0/12) | 8.21 ± 0.31 (0/12) | 0% (0/12) |
| (5) | | | |
| Rifampin | 3.68 ± 0.59 (11/24) | 1.43 ± 0.28 (19/24) | 46% (11/24) |
| (12.5) | | | |
| Rifampin | 2.14 ± 0.35 (15/24) | 0 (24/24) | 88% (21/24) |
| (12.5) + | | | |
| levofloxacin | | | |
| (5) | | | |
| Cpd 86 (3) | 4.76 ± 0.51 (2/12) | 2.77 ± 0.53 (4/12) | 8% (1/12) |
| Cpd 86 (3) + | 3.89 ± 0.32 (12/24) | 2.39 ± 0.72 (15/24) | 50% (12/24) |
| levofloxacin | | | |
| (5) | | | |
| Cpd 86 (12.5) | 3.24 ± 0.27 (5/12) | 1.57 ± 0.53 (10/12) | 58% (7/12) |
| Cpd 86 | 1.95 ± 0.34 (18/24) | 0 (24/24) | 92% (22/24) |
| (12.5) + | | | |
| levofloxacin | | | |
| (5) | | | |

^{b}(no. culture negative/total no.)
^{c}Cure is defined as absence of growth of S. aureus in the culture of removed cages (no. culture negative/total no.).

### OTHER EMBODIMENTS

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims. What is claimed is:

1. A method for treating a prosthetic joint infection in a patient in need thereof, said method comprising administering to said patient a rifamycin of any one of formulas (I)-(V) in an amount effective to treat said prosthetic joint infection.

2. The method of claim 1, wherein said rifamycin is selected from the compounds of Tables 1-4.

3. The method of claim 1, further comprising administering to said patient a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim, wherein said rifamycin and said second antibiotic are administered within 14 days of each other.

4. The method of claim 1, wherein said prosthetic joint infection is an infection of methicillin-sensitive and methicillin-resistant Staphylococcus aureus, Staphylococcus epidermis, Streptococcus spp., Enterococcus spp., Propionibacterium acnes, Peptostreptococcus magnus, Fusobacterium spp., Clostridium spp., Bacteroides spp., or Pseudomonas aeruginosa.

5. A method for treating infectious arthritis in a patient in need thereof, said method comprising administering to said patient a rifamycin of any one of formulas (I)-(V) in an amount effective to treat said infectious arthritis.

6. The method of claim 5, wherein said rifamycin is selected from the compounds of Tables 1-4.

7. The method of claim 5, further comprising administering to said patient a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim, wherein said rifamycin and said second antibiotic are administered within 14 days of each other.

8. The method of claim 5, wherein said infectious arthritis is caused by or associated with an infection of Neisseria gonorrhoeae, Staphylococcus aureus, Streptococcus spp., Enterobacter spp., Serratia marcescens, Borrelia burgdorferi, Kingella kingae, Escherichia coli, Propionibacterium acnes, Peptostreptococcus magnus, Fusobacterium spp., Clostridium spp., Bacteroides spp., Eikenella corrodens, Pseudomonas spp., Moraxella spp., Haemophilus spp., Streptobacillus moniliformis, Spirillum minus, Mycobacterium tuberculosis, Mycobacterium marinum, or Mycobacterium kansasi.

9. A method for treating osteomyelitis in a patient in need thereof, said method comprising administering to said patient a rifamycin of any one of formulas (I)-(V) in an amount effective to treat said osteomyelitis.

10. The method of claim 9, wherein said rifamycin is selected from the compounds of Tables 1-4.

11. The method of claim 9, further comprising administering to said patient a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim, wherein said rifamycin and said second antibiotic are administered within 14 days of each other.

12. The method of claim 9, wherein said osteomyelitis is an infection of S. aureus, Enterobacter spp. group A and B, Streptococcus spp., Haemophilus influenzae, Pseudomonas spp., or coliform bacilli.

13. An orthopedic implant which releases a rifamycin of any one of formulas (I)-(V).

14. The implant of claim 13, wherein said rifamycin is selected from the compounds of Tables 1-4.

15. The implant of claim 13, wherein said implant further releases a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim.

16. The implant of claim 13, wherein said implant is covered or coated in whole or in part with a composition comprising said rifamycin.

17. The implant of claim 16, wherein said composition further comprises a polymer.

18. The implant of claim 17, wherein said polymer is a biodegradable or a non-biodegradable polymer.

19. A medical implant which releases a rifamycin of any one of formulas (I)-(V).

20. The implant of claim 19 wherein said implant is covered or coated in whole or in part with a composition comprising said rifamycin.

21. The implant of claim 20 wherein said composition further comprises a polymer.

22. The implant of claim 21, wherein said polymer is a biodegradable or a non-biodegradable polymer.

23. The implant of claim 19, wherein said medical implant is a vascular catheter, prosthetic heart valve, cardiac pacemaker, implantable cardioverter defibrillator, vascular graft, ear, nose, or throat implant, urological implant, endotracheal or tracheostomy tube, dialysis catheter, CNS shunt, orthopedic implant, or ocular implant.

24. The implant of claim 19, wherein said rifamycin is selected from the compounds of Tables 1-4.

25. The implant of claim 19, wherein said implant further releases a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim.

26. A composition comprising a polymer and a rifamycin of any one of formulas (I)-(V).

27. The composition of claim 26, wherein said polymer is a biodegradable or a non-biodegradable polymer.

28. The composition of claim 26, wherein said rifamycin is selected from the compounds of Tables 1-4.

29. The composition of claim 26, wherein said implant further releases a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim.

30. A method for reducing or inhibiting infection associated with a medical implant, said method comprising the step of introducing into a patient a medical implant that has been covered or coated with a rifamycin of any one of formulas (I)-(V).

31. The method of claim 30, wherein said rifamycin is selected from the compounds of Tables 1-4.

32. The method of claim 30, wherein said implant further releases a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim.

33. A method for making a medical implant, said method comprising the step of covering or coating a medical implant with a rifamycin of any one of formulas (I)-(V).

34. The method of claim 33, wherein said medical implant is covered or coated with said rifamycin by dipping or by impregnation.

35. The method of claim 33, wherein said rifamycin is selected from the compounds of Tables 1-4.

36. The method of claim 33, wherein said implant further releases a second antibiotic selected from the group consisting of azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, carbapenem, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, fusidic acid, faropenem, polymyxin, tigecycline, AZD2563, and trimethoprim.

37. A kit comprising:
(a) a rifamycin of any one of formulas (I)-(V); and
(b) instructions for administering said rifamycin and, optionally, a second antibiotic, to a patient having a prosthetic joint infection, infectious arthritis, osteomyelitis, or a foreign body infection.

38. A kit comprising
(a) a rifamycin of any one of formulas (I)-(V);
(b) a second antibiotic; and
(c) instructions for administering said rifamycin and said second antibiotic to a patient having a prosthetic joint infection, infectious arthritis, osteomyelitis, or a foreign body infection.

39. A kit comprising
(a) a composition comprising (i) a rifamycin of any one of formulas (I)-(V) and (ii) a second antibiotic; and
(b) instructions for administering said composition to a patient having a prosthetic joint infection, infectious arthritis, osteomyelitis, or a foreign body infection.
